(19) United States
(12) Patent Application Publication
(10) Pub. No.: US 2012/0109241 A1
(43) Pub. Date: May 3, 2012

(54) Enhancement of Biological Functioning by the use of Electromagnetic and Magnetic Fields

(76) Inventor: Elizabeth Rauscher, Apache Junction, AZ (US)

(21) Appl. No.: 13/096,232

(22) Filed: Apr. 28, 2011

Related U.S. Application Data
(63) Continuation-in-part of application No. 11/837,397, filed on Aug. 10, 2007, now Pat. No. 8,062,229.
(60) Provisional application No. 61/349,740, filed on May 28, 2010.

(51) Int. Cl. A61N 1/36; A61N 1/34; A61N 1/362; A61N 1/40

(52) U.S. Cl. 607/9; 607/66; 607/65; 607/46; 607/45; 607/51

(57) ABSTRACT

Systems and methods are provided for treating a living being with multiple, concurrent, superimposed non-phase-locked signals, at physiologically acceptable intensities and duty cycles such that the signals entrain the tissue. Preferred signals are electromagnetic, and at least one of the frequencies is selected from the list consisting of 7.6 Hz +/−2 Hz. 70.25 Hz+/−0.25 Hz, 71.25 Hz+/−0.25 Hz, and 3040 Hz +/−10 Hz. Among other things, it is contemplated that the signals can be used to create a subjective reduction in pain, mood improvement, to treat osteoporosis, to enhance cardiac function, and/or affect the hypothalamic pituitary axis.

This application is a continuation-in-part of co-pending U.S. patent application Ser. No. 11/837,397 filed Aug. 10, 2007 and is a non-provisional of U.S. Provisional Application No. 61/349740 filed May 28, 2010, both of which are incorporated herein by reference in their entirety.

### FIELD OF THE INVENTION

The field of the invention is electromagnetic stimulation of a living body.

### BACKGROUND

Living beings react in many ways to extrinsic electromagnetic and other signals. Among other things it is know that some signals can cause or reduce pain, and can affect hormone levels, and various physiological processes such as digestion. What are not known are all the various parameters for achieving desirable effects.

### SUMMARY OF THE INVENTION

This application provides systems and methods for treating a living being, and especially humans, with multiple, concurrent, superimposed non-phase-locked signals, at physiologically acceptable intensities and duty cycles such that the signals entrain the tissue. Preferred signals are electromagnetic, and at least one of the frequencies is selected from the list consisting of 3.8 Hz, 7.6 Hz+/−2 Hz., 9.4 Hz., 70.25 Hz+/−0.25 Hz, 71.25 Hz+/−0.25 Hz, and 3040 Hz+/−10 Hz. Among other things, it is contemplated that the signals can be used to create a subjective and objective reduction in pain, mood improvement, to treat osteoporosis, to enhance cardiac function, and/or affect the hypothalamic pituitary axis and the purkinje process in the cerebellum and the endocardium.

### BRIEF DESCRIPTION OF THE DRAWING

A more complete understanding of the present invention may be derived by referring to the detailed description and claims when considered in connection with the following illustrative figures:

FIG. 1: The diodic antenna like system of the Purkinje Process that receives and transmits informational data from inside and outside the body and through other electromagnetically tuned signaling of the human body.

FIG. 2: Purkinje Fibers compared to other types of nerve cells. Note the richness of the antenna like pick up, receiving, and transmission systems.

FIG. 3: In the current view a wave of excitation from the S A or sinoatrial node traveling through the A V or atrioventricular node near the tri cuspid valve, then to the bundle of His and then to the Purkinje fibers, this comprises the mechanical excitation. In our model the electrical activity and stimulation proceeds from the Purkinje fibers back up through to the S A node. Mechanical excitation then proceeds back down as in the standard model. It should be noted that the terms “our” and “we” are used euphemistically herein, to refer to the inventor.

FIG. 4: Purkinje processes also reside in the cerebellum. The corpuscles of the Purkinje's are flask shaped cells with a large dendritic molecular gray layer array situated at the junction of the molecular and granular layers with their bases resting against the latter. The Purkinje corpuscles act as “diode” fibers or “cat whiskers” resting on the granular internal rust colored layer which acts as a semi conductor substrate. The granular layer rests the white substance.

FIG. 5: The Purkinje cell activity is displayed. Spontaneous cell firing is recorded for a 0.1 second trace end-to-end which shows a higher end frequency of about 70 Hz. This signaling is the upper of the two frequencies of 7.6 Hz and the approximately 70 Hz which is the nonlinear information fidelity step up frequency of the 7.6. Each frequency from the heart beat rate from the sinoatrial node, to the Purkinje fibers frequencies occupy a frequency range. Upper Purkinje cell frequencies entrain down from 70 to 7.6 to a 1.23 Hz pulse rate.

FIG. 6: Schematic of the flow diagram demonstrating the connection of the related body parts that are influenced and regulated by electromagnetic and electric type field communications.

FIG. 7: More detailed flow diagram of the electromagnetic like relationship of components, processes and operation of the vertebrate biologic systems.

FIG. 8: The hypothalamic pituitary (H-P) axis is a region of key regulation of and by external magnetic impulses of highly specific characteristics and internal voltage and current regulation acting as a receiving and transmission location. Coupling of the H-P axis goes from the 10th cranial nerve through C1 to C7 and from T1 to T4 and the cardiac region.

FIG. 9a: Denoted is the dominant DC (direct current), potentials of the human body.

FIG. 9b: Some frequencies having highly specific characteristics such as wave forms, duty cycles, and intensities that have physiological and state of consciousness on and within the human body. These comprise some of the many dominant biologic frequencies emitted by the device of this invention.

FIG. 10a: The generalized schema of the human body as an electromagnetic, electric and magnetic circuit involving various components of the body from the brain and heart, to the operation of cells, blood, bone etc. A cell is represented by a set of basic electrical component equivalents in a circuit that allows for a fundamental communication system.

FIG. 10b: The ATP power system of the cell is generated within the cell membrane. In FIG. 10b this process is displayed in which the protein bridges carry catecholamines which are normalized by the device of this invention.

FIG. 11: The output of the device of this invention is displayed in the time domain with a trace speed of a one second end-to-end. The amplitude is 0.2 V/div with an intermix of 7.6 Hz and 70.25 Hz is displayed from a three coil array. The resonant signal between 70.25 clusters is caused by the proper use of coil hysteresis in which the LC time constant is inductively coupled.

FIG. 12: This is the actual waveform generated by the device of this invention. The time domain trace is 10 msec. per division or 100 msec. sweep and the amplitude division is set at 2 volts/diva This time domain display is of the intermix of 7.6, 70.25, and 3040 signals.

FIG. 13: The spectral density from DC to 100 Hz is displayed with the intermix of three frequencies 7.6 Hz, 70.25 Hz (female), and 3040 Hz. The largest amplitude peaks are at 7.6 Hz and 70.25 Hz.

FIG. 14: The spectrum is displayed which shows the 3040 modulation and second harmonic from zero to 6.4 kHz.

FIG. 15: Displayed are the central pathways in the brain of some of the neurotransmitters of norepinephrine, dopamine, serotonin, histamine, and others that can be affected by electromagnetic and pulsed magnetic fields and can be normalized by the device, method and procedures of this invention.

FIG. 16a: The top trace is the emitted signal form the device of this invention. This trace is a 5 second trace end to end with no signal on. In the lower trace is displayed the corresponding brain waves of a 31 year old subject displaying eyes closed alpha wave frequency of 11.2 Hz., recorded from left frontal parietal, FP1 negative to cross hemispheres right occipital, O₂ positive and FP2 reference electrode. The amplitude is 100 microvolts per division for a 5 second trace end to end.

FIG. 16b: The top trace is generated 9.4 Hz. signal of 20 micro gauss per division from the device of this invention for a 5 second trace from end to end. In the bottom trace is displayed the Alpha brain waves of the sane subject as in FIG. 16A. Her brain waves are phase locked to the emitted signal and display an Alpha frequency eyes closed of 9.4 Hz. The same lead configuration is used and within less than 0.1 seconds of the previous trace, when the emitted signal was removed, her brain waves returned to an Alpha frequency of 11.6 Hz.

FIG. 17a: Dark areas are indicative of pain in a 47 year old, 5′10″ male of 185 lbs. The pain was described as continuous, shooting, sharp and throbbing. The patient had been in a car accident twelve years before treatment and had difficulty walking, standing or sitting. A number of unsuccessful treatment modalities had been used, such as an epidural block, an L4/L5 laminectomy, an L5/S1 laminectomy, and removal of bone spurs and scar tissue two years and eleven years after the accident. Since the car accident, the patient had not been free of pain and reported his VAS pain level at 8 to 9.

FIG. 17b: Treatment consisted of using the DC positive bias electrodes at C7 and negatively DC biased electrodes at L4/L5 and the sciatic injection points on the buttocks.

FIG. 17c: The second phase of treatment is to apply two out of phase negative coils to the sides just below the knee and just above the ankle. Each treatment session lasted for 25 to 35 minutes, for each coil configuration. His pain level was reduced from a VAS of 8 to 9 to 1 or less with 11 sessions over four weeks. He was almost pain free after a six month follow up for this difficult case.

FIG. 18: The pain reduction visual analog scale for Patient 5e is displayed whose pain and treatment modality is described in FIGS. 17a, 17b and 17c. The trend analysis is displayed as a least squares. Sessions were conducted for about one hour every three to four days. The VAS value points are denoted as a circle before the session and a square after the session. Six sessions were conducted in the inactive or “sham” treatment to which the patient was uninformed. The first active treatment was conducted on session 7 and thereafter with significant pain reduction for the pain originating from the L4/L5 region and significant sciatica. Treatment is more effective if the patient does not strain themselves during and after each treatment but this patient could not take time off from work. Follow up six months later yielded a tolerable VAS between 1 and 1.5.

FIG. 19a: This circuit diagram utilizes a voltage supply and frequency dividers to generate the 7.6 Hz signal having an approximately 50% duty cycle and the 70.245 Hz and 71.25 Hz signals, having an approximately 25% duty cycle. This circuit acts as an imprint to the circuits displayed in FIG. 19b.

FIG. 19b: The circuit diagrams display the triangle wave form generator for the oscillator frequency of 3040 Hz which is about 33% duty cycle backwards ramp wave. The intermix modulator of the three frequencies goes into the proper inductive lead to the coils which are applied to the patient.

FIG. 19c: The circuit used is a therapy count down timer and power switching controls for the device of this invention.

FIG. 19d: The circuit diagram for power management including regulators and battery charging.

FIG. 19e: The circuit diagram is for the implementation of the pain objectification of the device of this invention.

### DETAILED DESCRIPTION

I. The Body as an Electromagnetic System Coupled to the Methods, Systems and Device of this Invention to Reduce Pain and Enhance Health

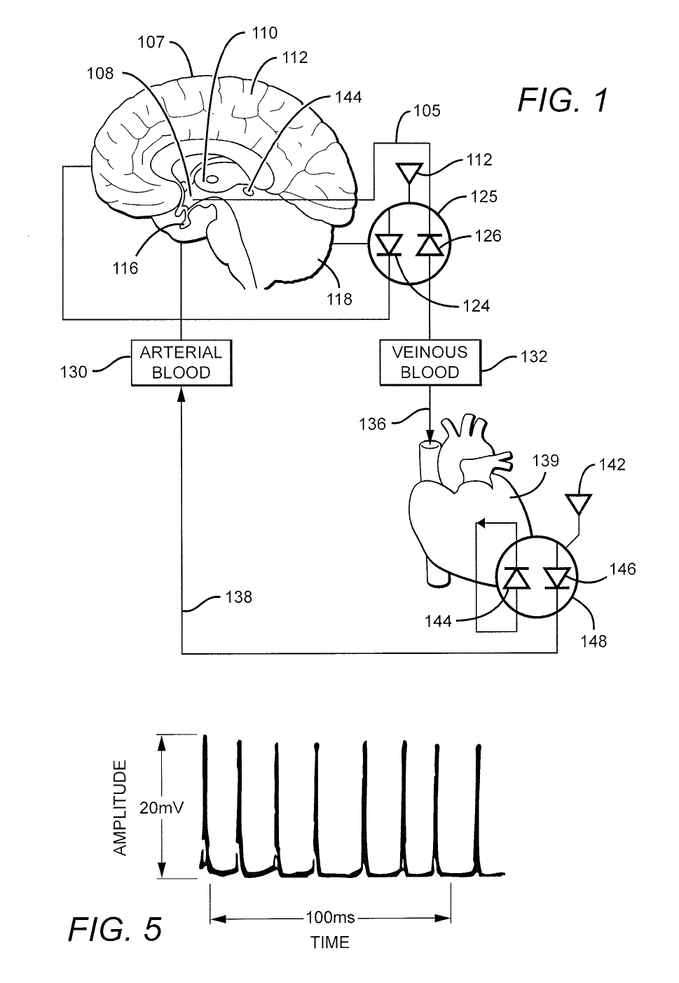

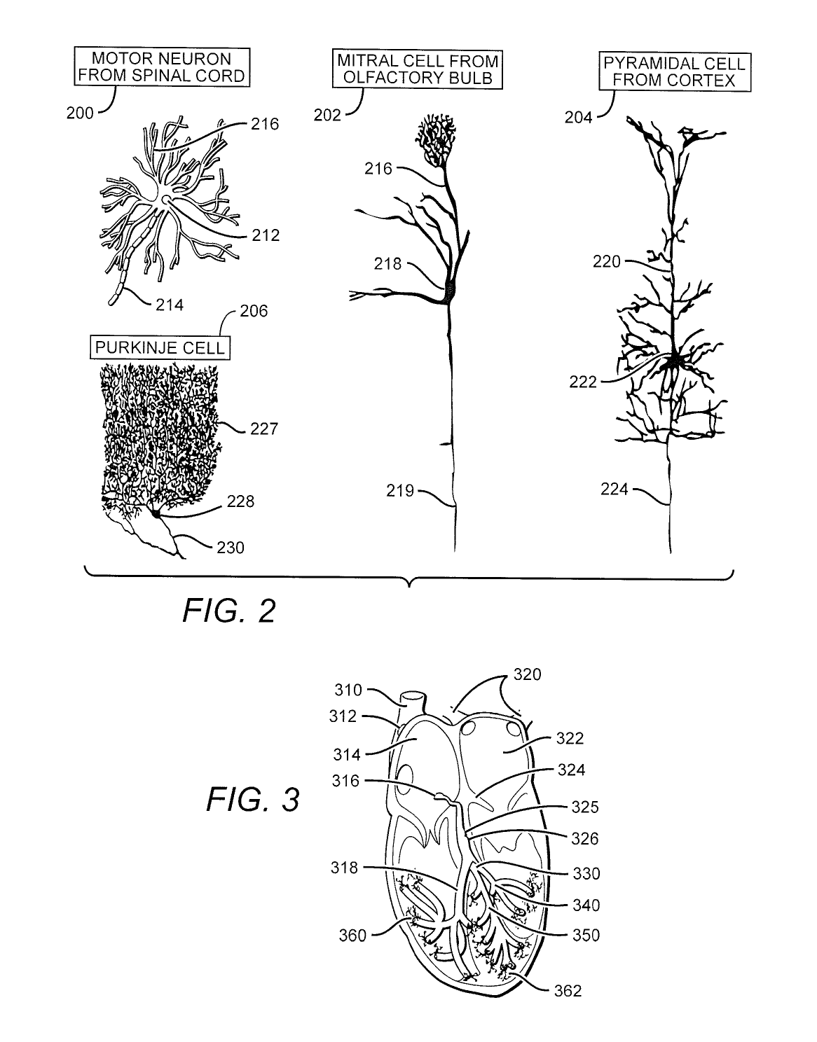

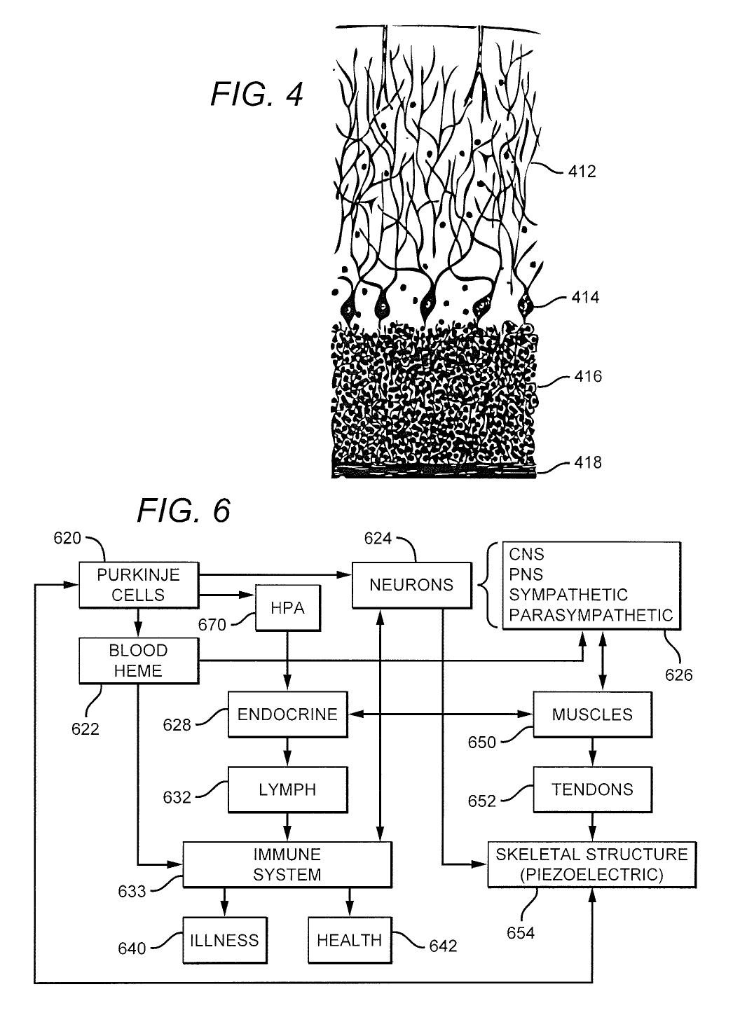

One can consider a human or other living body as an electrical circuit which is inductively linked to the electric circuits of the device of this invention. The output, through the coil emitters of the device of this invention, is received through the antenna receivers of the body. These receiving antenna like mechanisms operate through a number for systems such as through light, sound, skin sensation, smell or taste, but the key organizational processes for electric, magnetic, and electromagnetic fields is through the diodic antenna-like process of the cerebellum and cardiac Purkinje cells. See FIGS. 1, 2, 3 and 4.

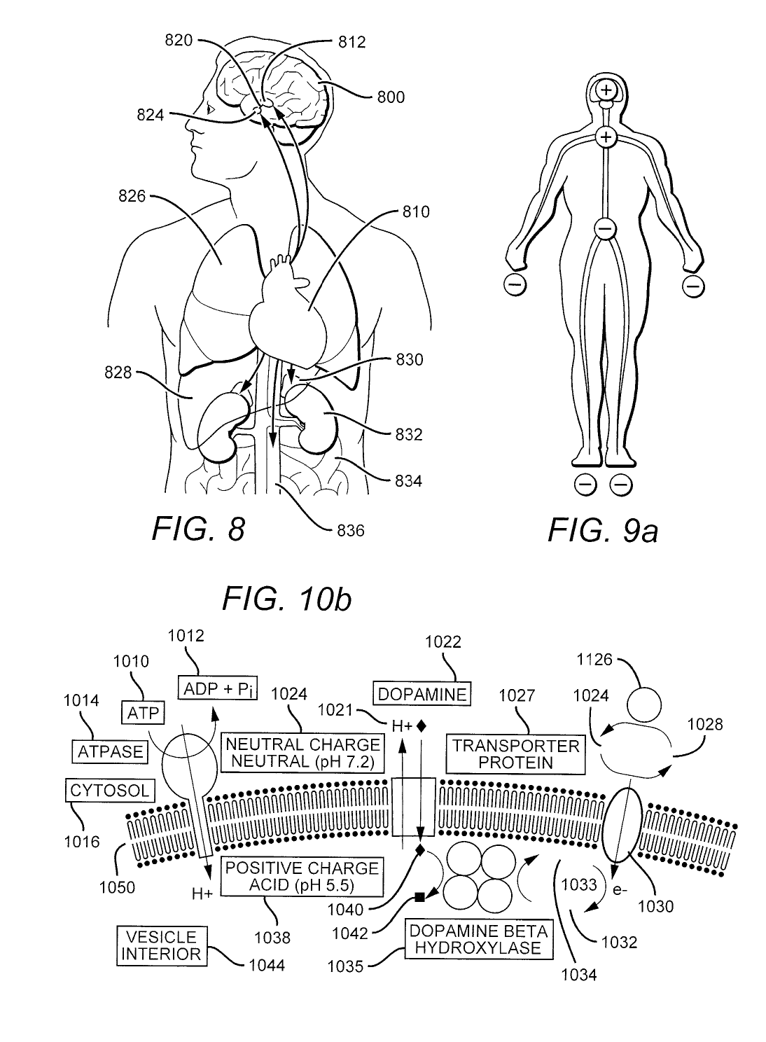

The cerebrum and cardiac Purkinje systems interact through CNS, PNS and autonomic nervous systems of the cerebrum and cerebellum and through the autonomic parasympathetic-paramagnetism of the blood which acts to record a bias in the vagus blood or nerve, which forms a feedback loop between these autonomic nervous system and the Purkinje rectifier system. This system both receives and transmits signaling between the brain and the heart primarily through the hypothalamic-pituitary axis and thalamus and pineal. See FIGS. 3, 5, 6, 7, and 8.

These systems, furthermore, are electrified through the cell membrane potential of all cells having a differential potential of approximately −70 mV between the cell outsides positive charge and the cellular inside negative charge which influxes potassium ion and effluxes sodium ion with the action of the chlorine ion and the doubly charged calcium ion. This charge differential is controlled by the semi permeable insulative membrane of the lipoprotein of the cell membrane which is resonant at RF frequencies of 100 GHz. this whole body system acts as the battery that runs and operates the whole of the body complex. Its operation depends on ATP metabolism and the “burning” of O₂ and emission of CO₂ and other products like an electromechanical internal combustion engine. This battery runs the whole of the biologic system. All cells organelles and organs are considered lumped LRC (Resistive, Capacitive, and Inductive) circuits. The whole body also contains the systemic paramagnetism of the blood throughout the body as is the internally generated structure of the bones current inducing piezoelectric effect and in striated muscle flexion. The neuronal cardiac QRS complex muscle and bone act as an FM-AM modulated like system which operates in the active and quiet state of the body and is modulated by variable capacitive and resistive electric elements of the body.

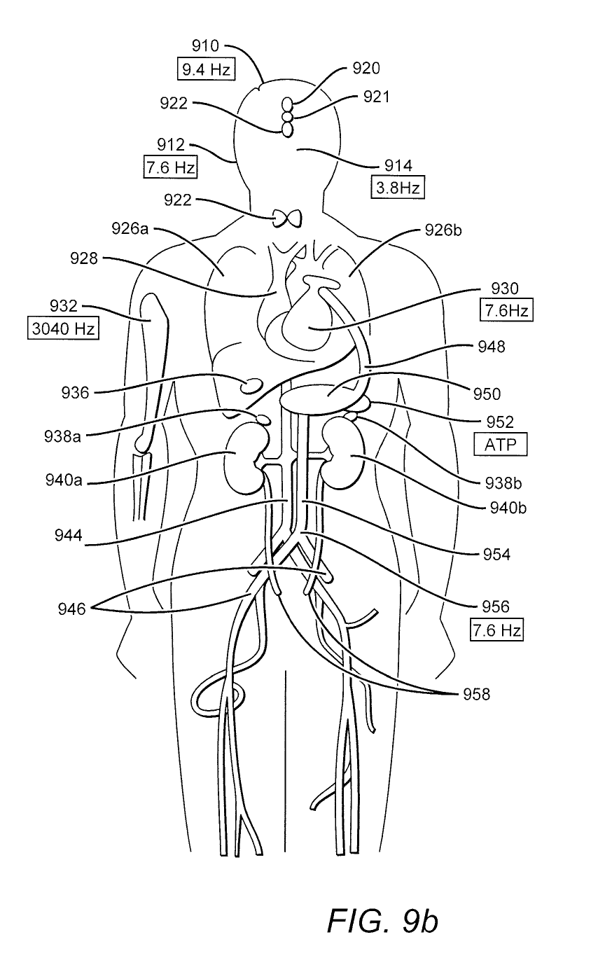

The activating and normalization factors of the device, system and method of this patent operate through antenna-like receptors in the hind brain and cardiac Purkinje process and through other major effects as influencing and affecting ionic permeability of membrane potentials. All the electrical and electronic systems of the human body are influenced, enhanced and normalized by the impedance matching and interaction of the device of this invention. The antenna like process of the Purkinje cells read external and internal bio-signaling and entrains and normalizes to the optimum proper signaling for electromagnetic, electric and magnetic fields having a frequency of 7.6 Hz. This is also the frequency of the hemodynamics of the iliac bifurcation. The aorta originates from the upper surface of the left ventricle and then descends down as the descending aorta, which is divided into the thoracic and abdominal aorta, where the abdominal aorta is divided into the two common iliac arteries at the iliac bifurcation. See FIG. 9b.

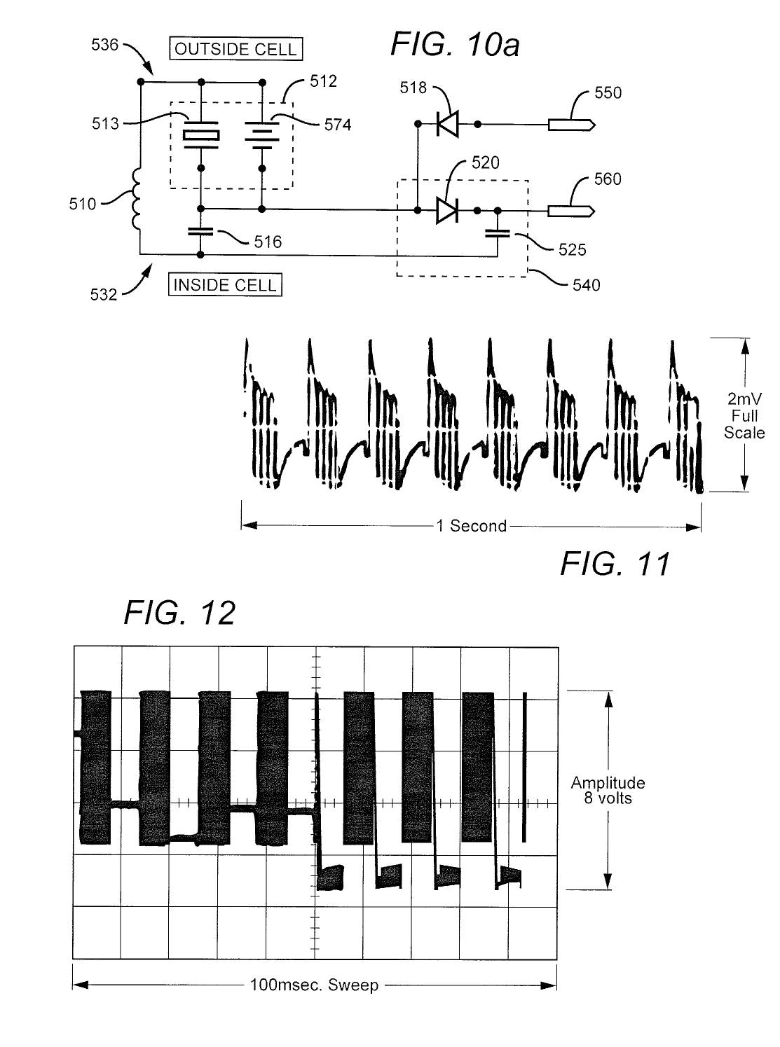

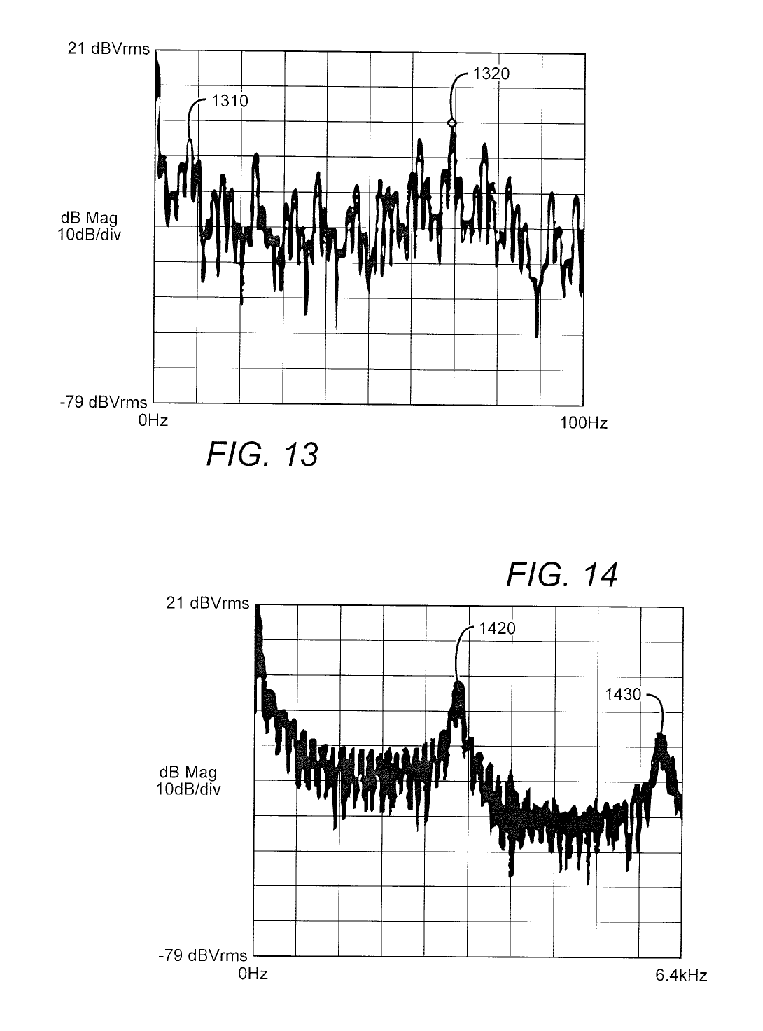

The hypothalamic-pituitary axis is one of the central processing units for the perception and programming of electromagnetic fields, anterior neuronal pituitary regulation, the release of hormones, Neurosecretory neurons serve as neuroendocrine transducers, converting neuro-information to hormonal information. Also involved are the endogenous peptides, endorphins and enkephalins. The sensitivity to pulsed magnetic fields of the hypothalamus, pituitary axis, as well as the adrenal-medulla, which is innervated by sympathetic neurons, is key to the pain reduction, relaxation and other modes of operation of the device of this invention. All systems of the human body and biological tissues in general, operate by electromagnetic signaling, potential differences and current carrying conductors. See FIGS. 10a and 10b.

The nonevasive pain reduction device and the body act in unison by means of the coupling of electromagnetic fields and the Faraday Effect of inducing currents between mediums or transforms one voltage to another. This effect transforms the signaling voltage content of the nonevasive pain reduction device directly into the cellular tissues and nerve fibers. See FIG. 10a.

A cell as represented by a basic electrical equivalent circuit consists of a number of devices that allow fundamental communications. As cells are selective to commands and stimulation, receptors, the analogue components can be described as a device similar to a radio transceiver.

Inductor (L1) and capacitor (C1), a tuned network, in the circuit FIG. 10a shows the communication method of detection of an electromagnetic stimulation. At the resonant frequency the coded signals of the device of this invention pass or are “tuned” to directly stimulate the cell at a genetic level. This inductive coupling also directly induces specific currents directly into the nerve fibers and the system. Inductor (L1) acts as the receiving coil and is coupled directly to the nonevasive pain reduction device by magnetic induction. The coils of the nonevasive pain reduction device create the transformer coupling or the transmitter.

ATP the power of the cell and the body is generated within the cell membranes. FIG. 10b shows the generation process, protein bridges carry catecholamines, protons and electrons across the membrane of the chromaffin vesicle. The enzyme ATPase breaks down ATP in the cystosol into ADP and inorganic phosphate (Pi), releasing energy that pumps protons (H⁺) across the membrane into the vesicle. A proton gradient is thereby created across the vesicle membrane: the pH of the vesicle interior is lowered, and the vesicle acquires a positive charge. The energy stored in the gradient drives the transport of catecholamines such as dopamine into the vesicle. As protons flow back out into the ctyosol a transporter protein carries dopamine into the vesicle. One step in the synthesis of adrenaline, the transformation of dopamine into noradrenaline, takes place inside the vesicle. It is catalyzed by a four part enzyme, dopamine beta-hydroxylase, with ascorbic acid as a cofactor. In the process the ascorbic acid loses an electron and becomes semidehydroascorbate. A third protein in the vesicle membrane, cytochrome b₅₆₁, transfers electrons (e⁻) into the vesicle from a complimentary process in the cytosol.

The bio feedback activity of the nonevasive pain reduction device and its derivative devices incorporate primarily two distinctively different synchronizing bio feedback techniques.

The passive feedback system is accomplished by driving the corrected nerve signals directly into the bidirectional nerve fibers, at a precise elevated level overriding and correcting any misinformation received by the brain, and similarly correcting the nerve signals to the therapy area. Cellular regeneration is stimulated at the therapy area by directed controlled induced voltages directed by waveforms produced by the nonevasive pain reduction device. Synchronizing in this fashion allow short preset therapy parameters to be administered in the structural active aspects of the body, for example bones, muscles, tendons and other bio mechanical active soft tissues. Passive feedback is primarily intended for acute pain reduction, and accelerated healing of bone and these types of connective tissues.

The Active Feedback system the effect of the nonevasive pain reduction device signaling can be monitored in real time and can be formatted for the specific treatment required. This will allow for adjustments of the nonevasive pain reduction device and its parameters and control many of the metabolic and biological functions within the body. These therapy techniques can be used long term and can have long term beneficial effects.

There are two major induction mechanisms to consider, electric stimulation can be induced deeply into bone and tissue by magnetic induction, exciting them at their resonance frequencies. Different cell structures respond to different highly structured frequencies that are delivered by the nonevasive pain reduction device. The spectral densities of the specific frequencies of the nonevasive pain reduction device are designed to interact in a precise manner with appropriate excitable tissues. Currents can also be induced directly into the conductive nerves system fibers by the same methods and topologies.

Selected Low and High Frequency RF carrier frequencies can be modulated and delivered by many methods. AM modulation of the carrier would allow the treatment frequencies to penetrate deeper. RF carrier techniques would allow a more focused therapy that could be directed by antenna or wave guide alignment.

The thalamus is the termination point for all the nervous tissue which passes through the twelve ventricles of the brain. The sensation of pain is perceived through thalamic processes and other processes of the human body. Pain can disrupt the healing process. Pain treatment and elimination, wound healing and other beneficial effects can be accomplished by application of electromagnetic induction through the human body by the device, system and the procedures of the device of this invention. The device utilizes the properties of biologic tissue to entrain and correct abnormal current flows and voltage potentials within the human body.

The beneficial effects of the device of this invention take advantage of the unique properties of the voltages and current flows controlling the operation of the human body. This is based on the fundamental fact that the body system will entrain to the most biologically correct internal or external signaling source. However, in the case of pain, injury and abnormal functioning of the internal system may not be able to correct and normalize itself but through proper externally applied signaling by the device of this invention, it will normalize to optimum correct external signaling.

Furthermore, the medullary neuronal complex is supplying 90% of its catecholaminergic innervations to the hypothalamus. Nerves in the medullary reticular formation project to the hypothalamus lined to noradrenergic and adrenergic pathways. Pain or noxious stimulation innervates the hypothalamus. Nociception through the A-δ and C fiber excitation stimulates the hypothalamus-pituitary-adrenocortical (HPA) axis which afferents information from other areas of the brain including the ventrolateral medulla and dorsomedial nucleus. Other afferents project from the hippocampus and amygdala which relates to the emotional and feedback cycle of Nociception. Links between the hypothalamus and autoimmune system and hormonal pathways are clear. Projections to the sympathetic spinal cord and parasympathetic vagal complex occur which release epinephrine and nor epinephrine from the adrenal medulla. These pathways are central to pain perception and the eradication of it. Normalization of the neurotransmission and hormones by the method, device and procedures of this invention is what allows and assists in long term lasting pain reducing and elimination effects. Also increases in the rate of healing occur by the device of this invention. The use of this device and procedures of this invention do not deactivate nociceptions to new current stimulation to avoid current injury, but allow these pathways to function normally. Also the blood carries the heme as well as its white cells which are part of the activation of the immune system.

Astrocytes in the dorsal brain account for hypersensitivity to pain. These can be affected by the method and device of this invention, so as to reduce the pain, and eliminate it and reduce the pain threshold. Modulation control occurs through ATP energy mechanism and many other factors including but not limited to interleukin 1β and the immune system. The device of this invention renormalized the patient's pain threshold and the associated biochemical such as Dopamine, histamine, adrenaline, noradrenalin, serotonin, endorphins, and enkephalins. The general scheme of this hierarchical organization of inner organ regulation process is the brain and heart ganglia. The brain and heart intrinsic auto regulatory mechanism proceeds from the cortex, the thalamus, the hypothalamus, brain stem, spinal cord and systems of the human body in general. Melatonin secretion also occurs in the hypothalamus and relates to the onset of relaxation and sleep. With pain reduction treatment, it is often noted that patients relax and can fall asleep during treatment. The hypothalamus frequency of firing is about 18 to 23 Hz, similar to the neuronal Ca⁺⁺ channel charge of 16-18 Hz. There are nighttime increases in the hypothalamus in the range of 20 to 45 Hz and lower frequencies of 1 to 18 Hz and a decrease of the 18-20 Hz frequencies. These frequencies are harmonics of the emission of the device of this invention.

Healing is produced through changing the threshold of the activation potentials and the Na⁺-K⁺ (Ca⁺⁺, Cl⁻) pump permeability to change and optimize them. The metabolite ATP is involved in supplying the all over universal battery energy system of the body. The pump is the active transport system of information and action. Precise duty cycles and wave forms of the 7.6 and 70.25 and 71.25 Hz signal used to normalize the CNS, PNS and autonomic nervous systems, is critical to the wound healing and reduced scarification of the device of this invention. Also, the process of white cell activation of the blood system and lymph, interleukin 1 and other systems relate to the healing-immune response of the body. Relaxation and normalization occur due to the device emitted 3.8 Hz and 9.45 Hz received by the body as its own generated fields to improve biologic functioning.

Cells neurons and organelles act in analogy to electronic components. Neurons are like coaxial conductors. The myelin sheath on nerves not only prevents current imprinted informational loss (analogous to i²R losses) but also shields against external extraneous currents. Myelin is the insulation that allows for reliable and rapid signal transmission. Glial Schwann cells provide a source of myelin and neurotrophic growth factors. The nodes of Ranvier act as junctures of myelin, like a lumped LRC circuit to accelerated the rate of current flow in the neuronal tissue.

The whole somatosensory process of sensorial modalities involves excitation or inhalation, conduction or insulation, afferent or efferent pathways which can be modeled as electrical conductive, resistive and capacitive diodic semi conductor substrate, all involving impedance matching. Some of these systems involve very high impedance of 10⁹Ω and lower impedance of a few ohms in which neuronal action potentials act. The processes are controlled and modulated and activated through electrical, electromagnetic, paramagnetic, magnetic properties of blood, heme and piezoelectricity of the bone, biochemical, chemical and mechanical processes in the human body. The body, when coupled to the device and processes of this invention, operates as an impedance matching net work to improve, enhance and normalize this symbiotic system.

Cells are separated by fluid-filled space like an “oil capacitor” or an electrolytic capacitor. A neuron is a cylindrical conductor which is a cable extending in one direction only. The input resistance is R_{In}=(R_{P} Rᵢ)^{1/2}/2πa³ and the resistance at the circular end is R_{c}=R_{P/πa}² where P is the parallel resistance, πa² is the cross sectional area. The current pulse is considered a square wave. For both cells, Rₘ=200,000 Ωcm. Rᵢ=100 Ωcm and a=10⁻³ cm. Then R_{P}=3.2Ω and R_{c}=637 mμ which is a high impedance of 0.64×10⁹Ω similar to the Rauscher-Van Bise Non-Superconducting Magnetometer, T-1050, of which the detector imprint impedance is 10¹⁰Ω. The impedance of the chest wall is about 1.4×10⁶Ω=1.4 MΩ. The cell membrane acts as a reactive capacitor and in a simple DC model C=q/V where C is the capacitive and q is the charged transferred from one conductor to another to set up a given potential, V. In a simple capacitor with two parallel plates separated by an insulator of dielectric constant, C, and thickness, x, and area A, the capacitor C=E₀ E A/d where E₀ is the polarization of free space which is 8.85×10⁻¹⁴ C/Vcm. Of course we find a more general solution for an AC system with DC bias.

The cell membranes are parallel plate conductors with specific capacities of about 1 μf/cm³ (micro Farad) equivalent to about 500Ω to 600Ω (such as used in the coils of the device of this invention). The pure lipid bilayer has a somewhat lower capacitive of 0.8 μf/cm² where the thickness of the insulating bilayer is 23 Å or 2.3 nm=2.3×10⁻⁷ cm, much smaller than a cell size of 10⁻⁴ to 10⁻⁵ cm. For the type RC net work representation of a cell, the input potential is V ˜−30 to −90 mV through a parallel resistant and capacitive which goes to ground. The change in voltage with time goes as dv/dt=V/RC and the solution to this first order differential equation has an exponential decay time of V=Vₒ e^{-t/τ} where V₀ is the starting voltage of t, with a time constant of τ for the membrane, m. This is related to charge and discharge rates where τₘ ranges from 10μ sec to 1 sec.

In the vertebrate system, the larger nerve fibers are myelinated which are lamella between the plasma membrane and extracellular fluid and have increased membrane resistance and lower capacitance. The myelin sheath is periodically interrupted by nodes of Ranvier which expose patches of axonal membrane. The node spacing is 100 times the fiber diameter or so and ranges from 2 m cm to 2 mm or 0.2 cm. Besides neuronal current loss due to increased resistance which restricts the current flow and hence i²R drop, excitation jumps from one node to the next, the thereby greatly increasing the conduction velocity on the leading edge of the action potential. Myelinated fibers conduct current faster, from 100 cm/sec to more than 10,000 cm/sec but are also capable of firing at higher, prolonged frequencies and periods of time.

The tank circuit's model of the cell can be applied to many other systems of the body. For example, the electric circuit representation of a fiber can be diagonal as a parallel and series circuit in which a power source goes into two parallel circuits, one having a capacitance, Cₚ and resister, Rₚ. The Rₚ is connected to the other side of the circuits in parallel to a series circuit having a resister Rₛ and Cₛ in series. See FIGS. 10a and 10b.

We can compare the electrical properties of voluntary or skeletal muscle, which are comparable to non-myelinated axons. Muscle activation times are of longer time duration than neuronal action potential time duration. The spike duration in the muscle is 40 to 200 cm/sec. In a simple picture of the Purkinje process, we can consider a tank circuit having parallel and series elements denoted by subscripts P and S, respectively, this model is a schematic to give a generalized conceptual framework. The analogous type of circuits for the Purkinje cardiac fibers has two time constants for only the DC part of the system. This system yields a net parallel capacitance, Cₚ and Cₛ for the parallel and series capacitance of the system. The action potential depends on the time constraint τ=Rₛ Cₛ where Rₛ is the series resistance with Cₛ. This time constant is an approximation as parallel processing occurs in the nonlinear biologic tissues. For the conductance τ=1/Rₘ and α=ωCₘ for frequency, ω, we have the trans-membrane impedance as Z=1/(σ+iα)=Rₛ+iXₛ where i=\sqrt{−1} The values of the capacitive Cₘ=2.4 μf/cm², Rₘ=1,000 Ω-cm² and Cₛ=7 μf/cm² have been measured. The time constant is dependent on the resting potential depolarization of ΔV, the change in the transmission potential as ΔV=iₛ Rₘ(1−e^{-t/τ}). The transmission voltage results from a current pulse which follows a resistive-capacitive RC charge circuit.

### II. Sources and Nature of Pain and Pain Treatments

Pain can yield useful responses to the system to produce avoidance such as from a biologic damaging source. However, pain can also occur such as chronic pain where the avoidance response is no longer useful. Also in current injury, during surgery and post operative care, pain can interfere with proper functioning of a person. Chronic pain not only leads to a reduction in the quality of life but can inhibit the healing process and lead to depression and suicide. Pain from severe injury can lead to shock and death and pain occurring during surgical recovery can interfere with the recovery process. Analgesics have been utilized but they just mask the pain and have side effects, sometimes major one, and often are not effective in treating chronic and other sources of pain and numbness and tingling sensations.

The invention combines the fields of physics, mathematics, engineering and biology to develop a new medical technology to control and correct excitable biological tissues and organs. The significant aspect of this invention is that it creates nonlinear dynamic techniques through practical instrumentation and device in order to imitate, mimic, entrain and improve biological functioning. The device of this invention is applied externally to the body and is coupled through magnetic field linkage from the instrument to the excitable tissue within the human and/or body and body organs. The emitted magnetic field from the biologic enhancement device interfaces with the biologic tissue, inducing current flow in the conductive tissue. This current flow is structured by the external magnetic field to match the body own normalized internal currents and voltages. Inductive linkage and tissue entrainment are fundamental to the operation of the device of this invention. Because the body operates as a complex of electromagnetic circuits with conductive and insulative properties, having the properties of an antenna, diodic processes, resistive, capacitive, inductive, energy source, magnetic and electromagnetic properties, it entrains to an externally properly inductively tuned system of the device of this invention. This system operates as both an analog and digital synaptic system with both AC and DC information and action modes operation. From ionic and polar molecular potentials, to cell activation and resting potentials, neuronal, paramagnetic (induced magnetic) properties of the porphyrin groups of heme to the hydroxyapatite piezoelectricity of the heme, the antenna like properties of the Purkinje process, to the energy power ATP battery, electromagnetic circuits power and operate the biologic system through biochemical potentials to produce a mechanical biologic complex of operations of the living systems.

The method and device of this invention utilizes completely unique, new and novel treatment methods that produce emitted complex fields that mimic and reproduce the body's own correctly functioning fields. When these fields impinge on the informationally sensitive tissue of the body, they are read and interpreted by the body producing signaling in the body which corresponds to the normal healthy state. Through the mechanism of this process, the device and procedures of this invention, by producing normal healthy signaling, reduce and eliminate pain. Unlike current treatment methods, which are often short term and ineffective, treatment with the device and procedures of this invention produce very long lasting results in pain reduction and produces no side effects. The pain reduction treatment by this invention is not felt and no unpleasant sensations to the person being treated and normal tactile and other sensations remain normal under the influence of the device of this invention

Treatment regimes depend on the source of the pain, the location of pain on the human body and, in the case of chronic pain, may depend on the years the person has been in pain. In some cases one treatment produces pain free conditions for months or years, for example, in the treatment of “tennis elbow” (lateral epicondyle of the humerus) or in another case, carpal tunnel syndrome, in other cases a multiple treatment regime are required. In the case of chronic pain, a patient's prognosis is not good once they have experienced significant pain over two years and the probability of their return to a normal life is minimal. With the treatment of the device, procedures and systems of this invention, over a relatively short period of a few weeks to a few months for treatment sessions which last from a minimum of less than 20 minutes to over an hour and a half, under usual conditions, patients have been pain free for ten to twenty five years from the treated source of pain. The corrected generated normalizing signals from this device is read and stored in memory through the afferent—efferent firings of neurons and in the hypothalamic-pituitary axis and other systems of the body. Although some surgical treatments for pain can be effective, most are not and in some cases the patient may be in more severe pain. This fact is assessed through multiple medical studies and study assessments presented later in section VG.

In vitro and in vivo studies, clinical studies and theoretical analysis, certain unique and highly specific frequencies, wave forms, duty cycles and intensities of electromagnetic and magnetic fields that interact with multiple types of excitable and reactive tissues in such a manner to improve biologic functioning to reduce and eliminate pain and treat the occurrence of some sources of pain. These signals from the external noninvasive source of this invention override and entrain the properly corresponding human tissues when transduced through impedance matching with these tissues. These experiments were comprised of human and animal in vivo and vitro studies and treatment.

The device of this invention incorporates new principles involving the ability of extremely specific external electromagnetic and magnetic fields to influence, affect and modify biologic tissue, organs, endocrine and neuronal processes. These new principles allow for bio-information transfer from a precisely tuned external imposed signal that mimics the corrected information for precise biologic functioning and transforms this information into the human body at precisely located points in order to eliminate pain. Pain relief occurs due to the renormalization of neuronal pathways in the brain, central nervous system (CNS) and peripheral nervous system (PNS) and acts to replace missing signaling, and correct distorted signaling in electrical biologic and other biologically active and responsive tissue in which pain receptor and other information signaling occurs. Medical terms are known to those versed in the state of the art.

When correct signal processing is applied to the human body, these signals entrain the proper corresponding signaling in the human body in such a manner as to produce correct and proper signaling in the human body. When this proper signaling is directed and driven in the correct corresponding tissues then pain reduction and elimination and other biologic normalization processes occur. The incredible sensitivity of biological systems to extremely low frequency and intensity magnetic fields of the properly tuned highly complex combinations of waveform, different frequencies, polarizations and intensities is of interest, not only because it will help physicians in the clinical setting but also help scientists to better understand information processing in living systems. One of the principles of the operation of the device of this invention is the requirement of the correct parameters of the signals of the device of this invention as overdriving and underdriving cannot work. That is, the correct intensity of the emitted signaling field is critical to proper operation of the device and method of this invention.

The device, procedures and methods of this patent description affects and allows normalization of neuronal pathways, replacing missing biologic information which is the source of pain and abnormal biologic functioning. As this process occurs pain is reduced by replacing the complex mix of Fourier frequency components in neuronal pathways and in the brain and other active tissues. Fourier components and analysis is known to those versed in the state of the art. The externally generated signaling effects deep tissue and is read by the body as its own signaling and the bodies neuronal and other biologic processes normalize to the correcting external signal which over ride, correct and normalize the body's own incorrect signaling. As this process occurs, endorphin and enkephalin production occurs and receptors are temporarily activated and then are renormalized, which reduces pain sometimes within about 20 to 40 minutes of the application of the device of said invention. Since the body's own analgesia is activated by the device of this invention no build up of tolerance or habituation to the treatment of the device of this invention occurs.

Further, long lasting effects of pain reduction and elimination have been achieved by the use of the device of this invention. Usually, as in our clinical and FDA trials, in severe cases of chronic pain multiple treatment regimes were required to significantly reduce pain. Treated patients who had been in “intractable pain” for over twelve years were pain free and were able to return to work and a normal life style and quality of life. Novel stimulants such as cold and heat and the use of the TENS, transcutaneous electrical neurostimulator have short term effects or no effects and produces sensations of their own and they only affect surface regions of the skin.

Chronic pain and some aspects of the pain mechanism are currently not well understood in the open literature. It is thought to be improper or incomplete communication between nerve cells. Other models such as nociceptor neuron or the substance binding model are not currently accepted. Pathways that transmit pain signals to the brain have not been well identified, so previous methods to block “errant” signals have met with no or limited success with unwanted side effects. We have developed an information model in terms of electric, magnetic and electromagnetic physiological response that involves neuronal processes, tissue conductivity, and hemodynamic, piezoelectric and other activity. It is clear to those versed in the state of the art that more than the usually considered neuro pathways is understood to be required to understand and treat pain especially chronic pain. When properly considered and treated pain can be reduced and eliminated by the device of this invention. Part of this process involves properly effecting the production and normalization of endorphin, and enkephalins which are the natural opiates produced by brain chemistry. The device of this invention also normalizes such neurotransmitters as serotonin, dopamine and norepenephine.

Pain is a sensation in which a person experiences discomfort, distress, or suffering due to provocation of sensory nerves. Pain can range from mild discomfort to intolerable agony. In most cases, pain stimuli, although sometimes necessary for warning about harm and proper reaction, are harmful to the body and tend to bring about maladaptive reactions by which the body attempts to protect itself from and results in reduced mobility, reduced functionality and reduced quality of life. Some physiological factors can be affected by the experience of pain such as increased blood pressure, suppression or imbalance of certain neurotransmitters, muscle atrophy, incorrect posture and many others. There are many types of pain described by the experience of pain such as aching, throbbing, sharp, stabbing, general discomfort, acute, agonizing, burning, cramp-like, numbness, tingling, debilitating, dull, and acute. Although the pain experience has always been with man, no viable theory of pain exists and no current treatment of pain without side effects from surgery and drugs. Surgery does not often resolve the problem and drugs mask pain and often have side effects including necessary continuous treatment and addiction and some patients may remain bed ridden.

Deep tissue penetrating transcorporeal fields from an emitter system are utilized to treat, reduce and eliminate chronic pain, such as arthritic and sciatic pain and pain due to surgical recovery, current injury and other sources of pain. Intractable chronic pain has been a nemesis to the medical community and to the patients themselves as no previous viable method of treatment currently exists. The device of this invention has been found to produce extremely successful treatment of pain with long term lasting effects without having side effects. An array of coil emitters are utilized in which coil emitters are located on the patient's body on the locus of pain and nerve injection points and other appropriate points on the human body. The optimum number of emitters has been found to be about six but in some treatment regimes less or more may be required. Field form is controlled by coil shape, wire size and shaped μ-metal core. Proper fields are emitted from an electrical circuit which induces pulsed biased magnetic fields from coil with μ-metal cores. The coils from the device of this invention emit fields which are read by the body as its own correct signaling and hence act to become like the body's own correct signaling mechanism.

The device and method of the invention operates such as to normalize the system as a whole. That is, it does not just interact only with the nociceptor functions, (of the current pain model) which are considered to be the whole of pain perception in the Somatosensory system. Noxious stimuli are thought to follow along pathways distinct from touch and pressure and are thought to be served by small diameter myelinated (conducting at 600-2500 cm/sec) and unmyelinated (conducting at 50 to 200 cm/sec) afferent neurons. The myelinated fibers are activated first and then the A-δ and C Reactive fibers. These processes do not act alone for pain perception or pain redirection, as many other systems are activated or modified or deactivated such as the spinal thalamic tract, thalamus, opiate receptors, etc. There are four types of neuron fibers having different electrical properties; Type A fibers are myelinated and have the largest diameter; type B fibers are narrower and have a thinner myelin sheath; type C fibers are small and not myelinated. Conduction velocity is significantly faster than its other types and the other spike duration of positively going voltage from the resting phase or negative polarity is longer in type C than in A or B as also the absolute refractory period.

All sensory stimuli, with the exception of the olfactory, are received by the thalamus, where they are integrated. The thalamus is the center for the fundamental sensations of pain in touch and temperature. Impulses are also received from the cortex, hypothalamus and corpus striatum and related to visceral and somatic effectors. The Corticothalmine connects the cerebral cortex and thalamus. In addition there are other processes that are involved in pain perception and also in paid reduction and elimination by the device of this invention. The hypothalamus lies beneath the thalamus and secretes inhibiting hormones. It is the main subcortical region of the integration of the sympathetic and parasympathetic activates. These systems also respond and normalize to the signaling injected and entrained to by the device of this invention. The pituitary gland is attached to the lower surface of the hypothalamus. The pituitary is an endocrine gland which secretes a number of hormones that regulate many body processes such as growth, metabolic processes and reproduction. The method, device and procedures of this invention specifically designed, based on our thirty years of in vivo and in vitro data and clinical trials to determine the specific frequencies necessary to utilize that reduce and eliminate pain, reduce inflammation, and enhance wound healing. Our detailed theoretical formulation gives a foundation for these design parameters and allows us to predict other critical frequencies to enhance biologic properties and lead to our engineering design features, The engineering design of the design and method of the device of this invention simulate the operation of the body and hence automatically produce the proper frequencies, wave forms, etc to enhance health. The basic principles of the theory are given in Table 1.

TABLE 1
MAJOR THEORETICAL PROPERTIES OF SELF-ORGANIZING
BIOLOGIC SYSTEMS

| | |
|---|---|
| 1. | Collective or mass motion of individual states, and coherent, |
| | non-dispersive low loss phenomena |
| 2. | Non-linear phenomena |
| 3. | Non-equilibrium open flux systems. |
| 4. | Coupled Resonance phenomena. |
| 5. | Dynamic or process-oriented phenomena. |
| 6. | Biologic information reception and transmission mechanisms. |

### III. A New and Novel Method Diagnosis and Long Term Pain Treatment

With the pain reduction and elimination with the device of this invention, no side effects have been noted in numerous clinical trials and long term pain relief has been effected. Patients that have been bed ridden over months or years have been able to return to a reasonably normal quality of life with the use of the device of this invention. The device of this invention not only does not inhibit healing but it enhances it.

Diagnostic techniques and medical histories are taken and are applied to determine the course of pain so that injury requiring repair and disease causes are treated when necessary. In some cases, immediate pain reduction treatment by the device of this invention can be implemented and others such as disease proper care are required and treatment for pain reduction is implemented by the device of this invention in such a manner to be consistent with the disease.

For example, acute and chronic back pain can result from trauma, injuries, and strains and is most commonly characterized by muscle tightness, tenderness or spasm and in some cases excruciating pain. Methods are employed such as X-ray, CT scan to diagnose and treat and eliminate possible fractures or fracture dislocations or herniated discs, nucleus pulpous which may require other types of treatments as well as treatments with the device of this invention.

Myofascial trigger pain determination and treatment with the device of this inventions can be applied immediately or after other treatment for fractures and during recovery of patients from debilitating back pain have been treated, using a correct series of appropriate treatments which use patients responses and the pain objectification device and procedures described herein. These patients have been able to function normally for many years after treatments with the device of, this invention. Some of the back injuries treated in adults originally occurred in childhood and treatment efficacy has been extremely effective with the device of this invention for old injuries or more current injuries.

A. A New Model of the Sources of Pain and Sensation

We understand pain to arise from lost information expressed in part as the lack of Fourier components which represent the informational component richness of normally functioning biological tissue. When these missing Fourier components are replaced by the device of this invention entrainment of the appropriate biologic tissue, then pain is eliminated and mobility returned. There is a direct correspondence between diminished mobility and pain. Increase of mobility to normalcy can be achieved when utilization of the device of this invention is applied. Pain occurs when incorrect direct signaling occurs which lack the proper frequency Fourier components and the information transmitted by these components. Also associated with some forms of mal adoptive biologic signaling is numbness or loss of sensation. This occurs when incorrect signaling, lacking a larger number frequency components of proper amplitude occurs, and the neuronal processing is incomplete. Anesthetics act by blocking nerve impulses as does numbing by the system itself, which involve a loss of information. Such processes are similar to but can be more extreme than that which produces pain but is the opposite process than that for rectifying pain signals which are eliminated by supplying greater information through more frequency components of proper amplitude from the device of this invention. Informational processing in all humans is the same. No specific receptors for pain have been definitely identified but there is the perception of sensations such as pressure, temperature, etc. What produces pain is the lack of viable information represented by missing frequency components in Fourier analysis and also low amplitude of the proper frequency components also is associated with pain. A new non-invasive, drugless and non surgical approach to chronic and other sources of pain will allow a new great advancement in medical treatment.

B. Pain Treatment Based on the New Model

The externally emitted field of the device and procedures of this invention will not only induce a reciprocity of inductive linkage field emissions from the biologic material it is acting upon but will also intermix with the body's own signaling mechanisms in order to enhance that system and reduce and eliminate pain. The interaction of the emitted received signals behaves as an imposed informational channel on the human body. The informational channel is a complex modulated frequency that then becomes an inductively coupled system. The manner in which the human body informational system operates is by the interchange of neuronal frequency variation modes or frequency modulation which require relatively low energy other systems require higher energy such as hemodynamic, musculature, collagenous and other systems in which amplitude intensity variation mode or amplitude modification modes which interact harmoniously, when a person is in an optimum state of health and free of pain. Neuronal and other conductive tissue is retrained to transmit “no pain” signals to the brain. FIG. 1 displays the time domain emission schematic of nerve, muscle and bone resonance tissue. Note that nerve impulses are short duration and bone is long duration and muscle lies in between.

Entrainment to the correct external informational signals reduces and eliminates pain and enhances physical functioning. Proper levels of neurotransmitters and hormones are found to occur when the proper emitter signals are used to entrain the neuronal and other pathways. The device of this invention reduces pain by returning the biological nervous system towards normal functioning. The key principles involved are magnetic fields that are generated at specific frequencies and the harmonics of those pulsed magnetic frequencies, wave shapes, direct current potential polarizations, produced by electrical currents emitted from coils will penetrate the system and stimulate specific and general nerve branches and nerve ganglion, and other parts of a biological system electrically, piezoelectrical response of collagen and of the bones, paramagnetism of blood heme and electrochemically, in concert when certain biological material coupled, resonant conditions are met. For the device of this invention a specific set of frequencies causes the ion flow in the nervous system of the human body to be efficiently moved along the nerve path where the locus of the pain exists and reduces or eliminates it. The device of this invention cannot be directly felt by the patient and hence double blind studies can be and have been conducted.

We have examined the frequency relationship of information which appears to hold for biological processing, in humans. We have examined the fundamental frequencies of the cardiac and blood flow system of about 7 to 8 Hz frequencies of the cardiac system and in the CNS and PNS set at between 70 to 78 Hz and for the CNS and brain there a three frequency bands, 1 to 40 Hz, 600 to 700 Hz and 1,200 to 7,500 Hz and for the piezoelectric resonance response we use frequencies in the 3,000 to 4,000 Hz range which can be set up to 8,000 Hz. Also it is noted that for the 2 to 5% duty cycle of the Hodgkin-Huxley model gives a frequency range of around 650 Hz. These frequencies are precisely set in the specific to the device of this invention operating system and are the same for all adult patients. The upper kHz ranges are approximately fifty times the CNS and PNS value. The respective frequency span for the cardiac, PNS and brain systems are 30.4 Hz, 281.0 Hz, and 7588.78 Hz. The beat frequency of the lower two frequencies of about 7.6 Hz and 70.25 Hz and 71.25 Hz is 22.8 and can vary between 23 Hz to 28 Hz for the beat frequency between 7.6 and 70 to 78 Hz. The intermix of the 7.6 and the 70.25 and 71.25 yields a sub harmonic of 3.2 to 3.8 Hz and a harmonic of 9.38 to 9.41 Hz.

A frequency modulation variation or AM like or FM like signal requires much less power than amplitude frequency variation and the FM signal requires, and does not respond to “noise in the channel” or misinformation when functioning properly so that signal rectification is superior for biologic information transfer, creating biologic functioning stability. The device of this invention's signaling behaves so as to give us a description of biologic neuronal processes that act in a self-organizing recohering manner utilizing the nonlinearity of biologic tissue. The device of this invention carries wave propagated information in such a manner as to recoherence dispersive losses of information. These informational channels operate as a resonant locked, low loss or dispersive free system. The beat frequency intermix is critical to the proper operation of the device of this invention. Thus, the modulator frequency is formed in such a manner so as to minimize informational losses and maximize the informational content in the informational channels, so as to remain relatively constant in space and time in the organs of the human body. We have measured in vivo these informational losses due to osteoporosis and injury which are observed as missing low amplitude Fourier frequency components. If there is disruption of conductive pathways as a result of injury or disease, there is a reduction in the informational content of neuronal impulses. The key to an efficiently operating frequency variation modulation system is that informational processes involve little change of energy of the carrier frequency. We give example output frequency displays of the output from humans and compare then to the output of the device of this invention.

C. Comparison of the Output of the Pain Reduction Device, and Biologically Enhancing System to Natural Fourier Emissions in the Human Body

Understanding the physical interpretation of informational system applications and biological description informational processing of pulsatile solitary wave solutions has come about only with our extensive and our recent research. We formulate nonlinear properties of biological informational processing in terms of pulse type eigen-functions. With this powerful formalization, we can deal with more complex, real world systems as opposed to the single neuron segment model of Hodgkin-Huxley based on work on the giant squid axon. The Hodgkin-Huxley (HH) signaling corresponds to single neuronal processes having an approximately 150 μsec duration activation this corresponds to the leading and trailing edges of a ringing square wave of about 650 Hz having about a 50% duty cycle and for the HH model which correspond to a non ringing square wave of about 5% duty cycle. In general, normal biologic functioning involves a number of neurons operating in consort to carry proper informational content. Neuronal depolarization occurs at approximately 6,000 mm/sec, whereas muscle response is more like 400 mm/sec, between 1,000 mm/sec to around 100 mm/sec. Hemodynamic flow rate is about 300 mm/sec to 4,000 mm/sec.

In our understanding of biologic process, two eigen function waves can cross each other's path and interact minimally where appropriate and hence allow little distortion or dispersive losses to occur. The phase velocity is proportional to the square of the amplitude of such a system. These waves are configured so that the nonlinear properties of the system overcome and “balance” the dispersive loss modes, to form a critically stable mode of the system. This is the manner in which biologic tissue information transfer operates and the device of this invention operates to induce corrected information transfer.

The manner in which the device of this invention effects biologic process in a beneficial manner are: (1) to match and correct biological signaling to corrected healthful states from an external impedance and bit rate matched source, to produce corrective entrainment of the properly corresponding biologic tissues, (2) long lasting effects are produced through corresponding correction of biological processes and effect-affect information transfer in conductive and reactive tissues, (3) corresponding neurotransmitter and endocrine system effect are produced by external corrective signaling (4) long lasting effects of a relatively short treatment regime is achieved due to the fact that the entrained tissues then re-emit their corrected signaling, which is reinforced by other tissues which are entrained to receive and transmit corrected signaling, and (5) deep tissue beneficial effects are produced by the deep tissue penetration of properties of magnetic fields. Proven efficiency of the device of this invention has been in numerous clinical trials and FDA studies.

### IV. Commonality of Frequency Response for All Humans

The optimum set of frequencies is the same for all humans with the slight difference such as 70.25 for females and 71.25 for males used in the device of this invention. The mal-adaptive systems informational pathways lack proper broad band and specific informational channels which can be rectified by replacing this information with an external source containing the correcting information. This correcting information is encoded into the proper set of frequencies which emitted and transmitted and transduced in the human body by the device of this invention.

A. Informational Processing in Biological Tissue

Neuronal pathways entrain and correct to the external signaling produced by the device of this invention. Proper hemodynamic function can be restored and piezoelectric response of bone and collagen also are activated so as to produce informational channels for pain reduction and elimination. Corresponding endocrine effects are produced by the external entraining fields in excitable tissues. The proper normalization of neuron transmitters and the endocrine system yields on objective measure of pain elimination. Also we have developed a system, method and device for pain objectification in the devices of the invention.

The key to utilizing corrective electromagnetic signaling, utilizing an external source of complex pulsed magnetic fields, is to match the corresponding informational bit rate, in responsive tissues, to magnetic signatures which induce current flows. There are three main methods whereby ELF and VLF signals can carry sufficient biologic information. They are: first, that complex signaling of the intermix modulation of higher frequencies modulating lower frequencies and second, the nonlinearities and recoherence of signaling allows a high informational hit rate, and third, the utilization of ringing square and triangle waves that carry higher bit rates, due to the great number of Fourier components.

A square wave can be constructed from a sum of add harmonics and a triangle wave can be constructed from the sum even harmonics. Note a sine wave, in its simplest form is associated with a single frequency and hence, does not occupy a significant role in biologic informational signaling as it is not complex enough to carry sufficient information. Integration over a frequency band of a square wave yields a triangle wave and the integration of a triangle wave in turn yields a square wave. The integral of even and odd harmonics yields a square wave. A ringing square wave generates dominate odd harmonics and a subdominant even harmonics. As these frequencies become higher, they descend in amplitude. It is the proper intermix of these specific wave forms which generate the proper information channel which is read by the biologic system as though it was correctly generated by properly functioning biologic or organismic systems.

Saw tooth or ramp waves are similar to triangle waves but due to the variation of their rise and fall times both have even as well as odd harmonics where the odd harmonics may be of lower amplitude then the even in the frequency domain. Ringing square waves, which are used in this application of this invention, will have even harmonics as well as odd harmonics in which the even harmonics may appear of lower amplitude in the frequency domain. Ramp waves are also used. Beat frequencies and frequency intermixes created by multiple frequency sources and wave forms and their respective harmonics must be constructed in such a manner as to be highly specific to form the proper responsive resonance with the field sensitive biologic tissues. These wave forms are known to those versed in the state of the art.

The reason the device of this invention can operate so effectively is that the tissues of the human body which are responsive to externally generated proper biologically response informational channels in the human body are similar in form, frequency and intermixes of these waves and or interpreted by the body as though they were generated inside of the human body when so properly generated. Biological systems are self organizing, nonlinear, not at equilibrium and act as an energy driven flux system which is dynamic and process oriented. Systems and subsystems are informationally locked in order to form the dynamic status for organism viability and optimum functioning. The externally generated fields of this device produce those which match to normal healthy tissue functions and these tissues functions and these tissues entrain and correct naturally to these external fields.

A number of systems, organs, organelles, cells and biologic structures and substances of the body are affected through pulsed magnetic fields which can produce current flows in conductive soupy tissues. These current flows as well as hemodynamic effects of paramagnetism produce proper signaling in healthy systems. When chronic pain or other sources of pain exist, abnormal responses are produced in the system. When current injury pain warning systems are no longer necessary and pain can lead to mal-adaptive functioning, shock or chronic debilitating pain and reduced quality of life, then the device of this invention can produce corrective normalizing effects from the external sources of the device of this invention to produce corrective effects. Central to the understanding of informational processing in biologic tissue involves the collective, coherent behavior of biologic tissue as well as its nonlinear properties. Increased efficiency of biologic tissue occurs because it is for from equilibrium. All biologic processes involve dynamic process-oriented phenomena which recohere dispersive losses when these tissues are operating properly. Under these conditions, maximum energy and information is retained.

All biologic processes are energy flux processes whereby energy, information, electromagnetic, mechanical and chemical process influx and efflux through and by such systems. This is critical to their proper operation. It does not matter to the processes and operational functioning of such a system, whether the energy, informational flux originates in that biologic system or from an external source both are read by the system in the same useful manner to said system. All biologic tissue is designed in such a manner that is responds to the optimally designed energy and information influx as to function with the least amount of dispersive losses, the lowest informational and energy entropy conditions and the optimum state of functioning of the biologic set point. Hence, if externally generated signals impinge upon and penetrate biologic tissue so as to create the conditions for an optimally functioning state of that system, then the system will override its own internally mal adaptive set point signaling. Any erroneous, incomplete or incorrect internally generated signals to recognize, match and self reproduce the externally generated impinging correct signals in such a manner as to correct its signally to the optimally functioning state. Key to the operation, effectiveness and utilitarian ethicacy of the device and procedure of this invention is the fact that not only does biologic tissue read and correct itself to the proper signaling but it also will maintain its state of normal optimum state of functioning for long extended periods after the treatment and use of the device of this invention.

B. Electromagnetic Field Signaling in Various Systems of the Human Body

There are many systems of the human body that utilize electromagnetic informational signaling. The higher range of informational bit rate data is processed in the lipoprotein cell membrane layer which can act like a solitary wave propagator, cohering signal processing on the cell membrane and between cells. The lipoprotein layer acts as a high frequency transmitter and receiver of 3 mm electromagnetic transfer information. This is one of the very high end of biological range of frequencies, outside of inter cell nucleus. The lipoprotein cell membrane acts, in part, as a rectifier as do other tissues of the human body having a high associated bit rate. The equivalent circuit is two tank circuits which are both capacitive and inductively loaded, having resistance.

Afferent and efferent firings of the sympathetic and parasympathetic nervous system operate around 10 Hz and are controlled, in part, by the vagus nerve, which is connected to the hypothalamus and mutually operate to relay information between the brain and the body. The vagus nerve connection to the hypothalamus acts as a direct connection to the afferent—efferent process and acts as the parasympathetic channel to the Purkinje process in the pons Varolii. The 7.6 normalized cardiac frequency is set as the lowest frequency on the step down “transformer” conversion process in the indirectly linked processing the blood pumping through the iliac bifurcation sets up an approximately standing wave of 7.6 Hz which is a fundamental hemodynamic oscillatory frequency in the body. This signaling affects the cardiac endocardium and the cerebellar cortex and the white matter of the cerebellum having a signal rate of 0.132 sec.

C. Signal Processing Modulation of the Human Body

The method and device of this invention utilize AM-FM modulation, digital and analog (primarily analog) processing and DC and AC signaling are all required to enhance biologic functioning. This is because the electric and electromagnetic mechanism of the body operates upon these principles.

1. Digital and Analog Processing

The analog systems of the human body regulated the digital neuronal informational processing of the human body. Synaptic digital like signaling and muscle flexation, which emit analog type signaling exist in biologic informational processing. Information can be transmitted bi-directionally or multi-directionally. Digital biologic signaling can act as a one way grating such as in the synaptic cleft moving neurotransmitters to receptor neurons. This process is analogous to diode gating. The channel frequency modulates the interacting system with a forward—reverse voltage flux. Thus this channel acts as a frequency modulator.

Each process of the body operates by the transmission of energy or traveling through a given self resonant system. The resonant system will then be stimulated to respond with a transmission of its own, which is characteristic of that particular system, activated by the impinging fields form the match resonant source. It is this principle of operation of biologic signaling of locked resonant behavior that allows the device of this invention to operate effectively on biologic tissues to normalize and enhance its processes. That is, since each system of the biological whole is resonantly locked in an informational manner, each subsystem detects these signals from other parts of the system as external and hence our device can take advantage of this type of biological operation in such a manner that the use of a properly operating external signal is read by each biologic subsystem as the proper signaling and will naturally entrain to the most optimally correct signal even if from an external source. We take advantage and utilize this fundamental operation of biologic functioning. All biologic tissue entrains either internally or externally to the most optimum natural form of signaling and hence this is the basic principles upon which the device of this invention operates.

All biologic processes are analogous in nature. To interact with and normalize the functioning of the body, the device of this invention requires an analog signal processing. Some processes can be mimicked by digital processing but the key to the design and operation of the device of this invention is the proper variability in capacitive resistive and inductive properties of the circuits of the device of this invention. Some biologic processing can be considered in part, digital-like such as neuronal activity.

As in the case of neuronal activity sometimes neuronal pathways transmit signals and sometimes they do not at their synaptic cleft or junction. One of the keys to biologic functioning is the enhancement of the signal to noise ratio at the collective neuronal activity level, catecholamine release increases the responsively of cells to excitatory and inhibitory inputs improves signal detection and performance as a whole. Based on the principals of parallel processing, and at the informational processing level, catecholamine affect the ability to detect signals when they are imbedded in noise. Release of norepinephrine and dopamine occur over wide areas of the CNS and the post synaptic effects of the release of these catecholamines. One important effect consists of an enhancement of target cells to other afferent inputs, inhibitory as well as excitory This action occurs in analogy to the operation of a transistor which is a semi conductor which allows and controls electrical signals to flow in one direction or to be rectified. Zener diodes for example, blocks current flow completely in one direction and blocks it in the other direction until a threshold voltage is reached this is analogous to neuronal transmitter action in the synaptic clef which act as a digital like gating process. This on-off pulsed system can be considered a DC digital-like pulse in the neuronal junction. It is not truly digital and the whole of the biologic processes operate in an analogous manner.

Long pulse trains are created by neurons (narrow pulses) and muscle contractile response (wide pulse) and other biologic tissue. Diodic-like process in biologic tissue can act to convert AC signaling into DC signals. The biologic AC signaling processes throughout the system collectively sets up the DC bias found trough the body.

2. Modulations of Electromagnetic Signaling Through a DC Biased AC System

A frequency modulated system requires much less power than an amplitude modulated system. The frequency modulated system is much less responsive to noise sources, static or other extraneous signaling either internally or externally generated and is hence superior for biologic information transfer such as in the human neuronal pathways. The frequency modulated system is carrierless because it modulates the carrier completely. In fact, the response of such a signal, with its information stability, has a good analogy to the Josephson junction super-conducting phenomena. This analogy gives us a description of biologic neuronal processes that act in a self organizing manner like an “ambient superconductor”. The carrier like frequency modulated signal is approximately 70 millivolts cell resting potential.

A balance of charge exists on the inside surface contiguous tissues and on the outside as a “digital” action potential which propagates along the neuronal axon. The outside and inside potentials maintain an opposite charge having a negative action potential of 70 volts and a positive potential of +40 volts. An isolated nerve fiber, such as that of the giant cylindrical axon of the giant squid, maintains a constant potential difference called the resting potential across the surface membrane. Above the threshold potential, current signaling is transmitted. After the occurrence of the signal transmission, the nerve fiber returns to its threshold resting potential with a relative refractory period nerve cells can act as one way informational gates. Properly design externally generated magnetic fields can effect and normalize the set points of these electrical potentials.

Both time varying AC and time constant DC current potentials occupy a fundamental role in biological systems and in humans as does digital and analog signal processing. The AC and DC component potentials measured on the skin reflect the configuration of the nervous system as well as other current carrying tissues. For the DC component, the positive potential is that of the main nerve ganglion, the human brain and also the upper part of the spinal region, ending in C7 is primarily positive in electrical potential. The spinal region, with its great concentration from the brachial plexuses between the shoulder blades and down the spinal column to the L1 to L5 region and the sacrum is negative in electrical potential. The limbs carry a primarily negative potential. These facts are key to the operation of the device of this invention as the primarily AC pulsed magnetic fields of this invention are based in a DC manner such as C7 utilizes a plus based reference electrode and the links, lower back and other areas require a negatively based DC electrodes and the limbs are treated with DC biased electrodes that are negative.

The nerve signal travels along the axon as a pulse potential and is rectified at the synaptic cleft to produce a digital like signal. The synapse or junction point between two neurons do not come into direct contact but bursts or neurotransmitters produce the digital connective link between them, carrying the information across the synaptic cleft. The neuron axis carries a constant potential with constant current potential with almost no dispersive losses. Analogy to a superconductor has been made but a more viable model is that of a nonlinear segmented lumped LRC tank circuit which has nonlinear recoherence factors that overcome dispersive losses. Thus this system acts as a low loss or no loss system. Such a system is highly susceptible to small external properly timed signals as nonlinear, high information carrying current potentials. Such potentials are induced from internally correcting electromagnetic signaling or when this signaling is mal adaptive, correct signaling can be introduced from external source such as the correcting induced current pulses from the device of this invention which introduces signaling that overcomes dispersive losses and replaces lost or incorrect signaling. The effects of the device of this invention have long lasting effects because biologic systems, once properly entrained, remain so if not otherwise affected by another injury, etc.

The basis for the theoretical approach is the consideration of nonlinear, collective, coherent coupled resonant phenomena, which can be electronically represented as a lumped LRC tank circuit. The nonlinear form of differential equations gives coherent, non-dispersive solutions. The solution to such equations is eigen functions and these equations admit certain extremely stable conditions, having low dispersive losses. The analogy of neuronal axons to insulated wires is obvious. Most axons are surrounded by a discontinuous myelin sheath which consists of many concentric layers of membrane. The myelin sheath is an effective electrical insulation; the myelin sheath of neurons in the CNS is formed by oligodendrocytes and in the PNS by Schwann cells. Myelination decreases the axons capacitive and conductive leakage permitting a depolarization event to spread faster than along a non-myelinated axon. For depolarization to spread from one site to the rest of the neuron, the action potential must still be renewed periodically down the axon which occurs at the nodes of Ranvier. The nodes of Ranvier are the only locations on the myelinated axon where the action potential can be generated because they are where the voltage gated sodium channels are concentrated. These voltages gated chemicals move along nodes a discrete jump, not a steady ripple, and hence are highly subject to externally generated properly pulsed fields with large information content as produced by a large number of Fourier frequency components emitted and received in these junctions from the device of this invention. When not correctly operating, neuronal and other processes have dispersive losses, which lead to pain and other medical problems. Replacing this missing information and overcoming dispersive losses, rectifies these processes and pain reduction and other corrections occur.

The AC component of biologic signaling process is complex and utilizes wave forms that generate the required frequency components. In general most biological signaling analysis utilizes the time domain such as for electroencephalograms EEG and electrocardiogram EKG. These measurements are dependant as is GSR on measurements of the ubiquitous current flow of charge carriers, and potentials generated in the human body. Frequency and time domains, for those versed in the state of the art, are related in an inverse relation. In its simplest form f is 1/t and f is frequency in Hz, and t is time in seconds. In our application, this relation is much more complex and is expressed as a dispersion relation. As the span of frequencies, in the frequency domain goes to infinity, we create the time domain. Span and band width of frequencies are relevant to producing a specific biologic effect for informational signaling.

We describe biologic process in terms of analog and digital processing and operating as a DC biased AC pulse modulated information system. Neuronal systems primarily act as a high information resolution low energy input FM system whereas muscular cardiac pumping action, digestion and elimination as well as sex, act as AM large variation amplitude on a large energy input system. Neuronal FM systems have their operation of a lower energy feedback system and their larger movement and actions involve the AM system. The device of this invention operates using both digital-like and analog signal processing as does that of the human and other animal bodies. Digital signaling occurs in single pulses or a pulse train discernable in terms of individual digital bits of information. For example, this type of signaling is associated with neuro-transmission across the synaptic cleft as an on-off signal. Analog signaling involves a continuous signal that changes in time by various changes in signal strength or intensity and changes of form in space and time.

Simple sine, triangle or squares occur in nature. The body utilizes primarily a complex intermix of various forms of square waves and triangle waves. Sine waves have little or no effect on the body emitted from an external source such as by the material and device of this invention and are not utilized by the body as they lack the complex signaling components or harmonics necessary for sufficient information transmission within the body. Analog AC signaling can be zero DC biased or contain a DC offset or non-zero bias. The concentration of the largest nerve centers, the brain and the region of the brain are positively DC biased while the lesser concentration of nervous tissue, and nerve endings in the body, are biased negative. See FIG. 9A.

Charge carriers can be rerouted or pulled off a diode system by an external strong magnetic field. Hence current flows and gating may be transformed. In general the field strength of the device of this invention can produce ionic current flows that activate dormant gating effects. These semi-conductor diode gatings primarily occur in the neuronal processes. These neuronal systems utilize and respond to form DC bias but their impulsed informational transmission utilizes a pulsed AC system. See FIG. 9b. Both of these operational modes are utilized in the design and operation of the method, device and procedures of the device of this invention. These gating or semi conductor properties occur in the brain as in the CNS and peripheral nervous systems. In the brain there are billions of nerve cells but also billions of perineural cells, the most common are glial cells in the brain and also in the spinal cord. Schwann cells encase the peripheral nerves. Originally perineural Glial cells were considered neuronal tissue “glue” but can act to generate their own electrical potentials and appear to also participate in electrical and magnetic body communication systems. It has been hypothesized that it is the perineural cells and the Schwann cells that carry the electrical signals to heal bone fractures. There is a signal spread in the DC carrier signal observed as the evoked potential response one half second before the muscular action which appears as a FM system acting with side carriers. This evoked response is a readiness potential which suggests an electrical system with DC bias that voltage modulates the components as the FM carrier for the nervous system. For this system to operate between FM and AM, The nerve-impulse system has an electrical state that is ready to trigger the events of the thought moving to action. The data transmission and control mechanisms are composed of an electrical, magnetic, and electromagnetic control systems. The DC electrochemical potentials are the energy battery power sources of the FM like inter-communication system.

3. AM and FM Like Biologic Modulation

The human body and living systems in general are extremely complex, interactive dynamic informational energy exchange systems within the system and between the system and external process through the phenotype. A major component of the process of energy-informational transmission and reception is performed by electromagnetic forces. These forces have highly specific frequencies, wave forms, intermix modulation and intensities to key into and control the proper components of the complex array of operational biodynamic processes just as in the case of biochemical processes, extreme specificity of operation is necessary for optimal functioning. The human informational channels in the brain, CNS, cardiac, hemodynamic and piezoelectric systems: particular emphasis is given to the Purkinje cell pacemaker process which acts as a receiving antenna and on internal antenna processing communication within the biologic system. The pineal gland which is sensitive to the electromagnetic spectrum near the occipital lobes relates to daylight and night time light and RF absorption and perception. This is an entry way for electromagnetic effects which coupled with the adrenergic and cholinergic pathways and active-inactive periods of biologic activity. The major cholinergic pathways operate from the medial septal nucleus, the nucleus of the diagonal band and basal nucleus. Effects of certain specific physiological pathways are more responsive to the electromagnetic emissions and receptions from external and internal stimuli.

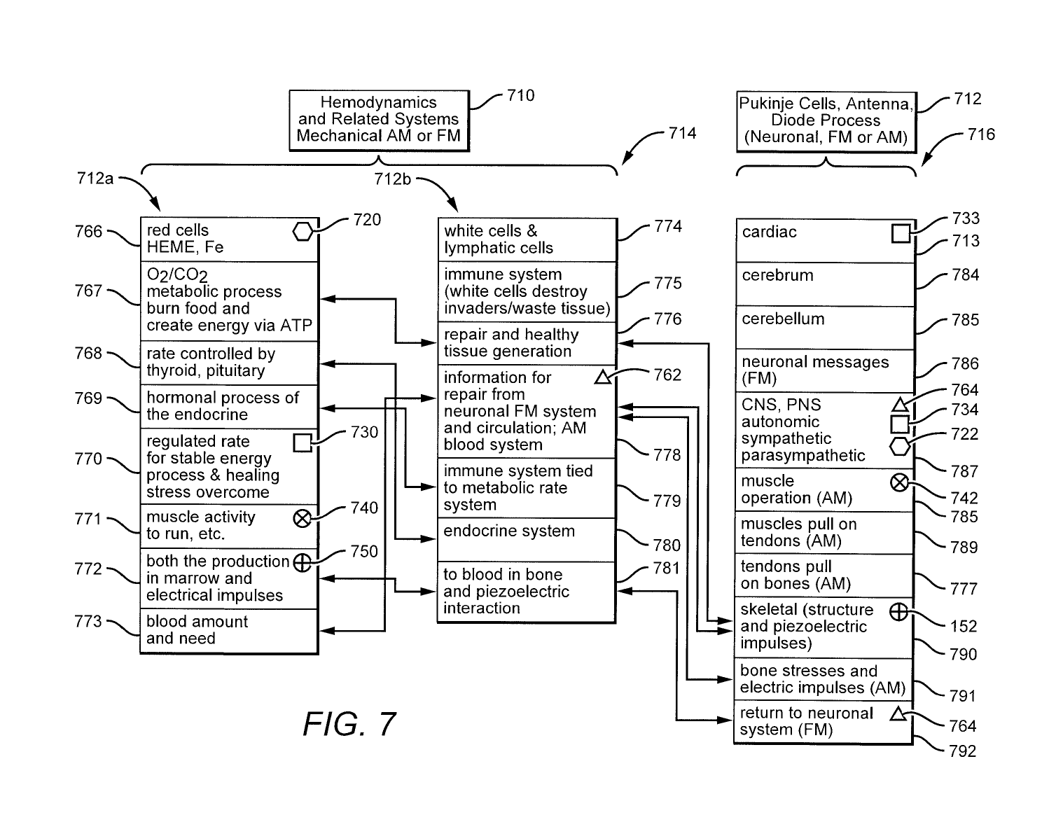

A frequency modulated FM signal requires much less power than an amplitude modulated AM. The FM does not respond to static and interference as much so that signal rectification is superior for biologic information transfer. In fact, the response of such a signal, with its information carrying stability, forms an analogy to the Josephson junction. This analogy gives us a description of biologic neuronal processes that act in self-organizing and recohering manner, like a soliton transmission line system. the carrier-like FM signal powered by the approximately −70 milli volts internal cell D.C. bias resting potential. See Table 2, and FIGS. 6 and 7.

TABLE 2
INFORMATIONAL CHANNELS:
OUR MODEL OF INFORMATIONAL CHANNELS IN THE HUMAN BODY AND THE
OPERATION OF THE DEVICE OF THIS INVENTION

| | |
|---|---|
| 1. | Hydrodynamic “Standing wave” or soliton modes in the blood-cardiac system which |
| | comprise one of the informational channel in the human body. This channel operates by |
| | encoding magnetic field information utilizing the paramagnetic properties of the heme. |
| | Standing wave formation occurs in the iliac bifurcation of about 7.6 Hz. |
| 2. | Electric flow occurs as an informational and transformational channel in electrolytic |
| | saline solutions in the human body Electrochemical and biochemical process can be |
| | induced by magnetic field impulses and carry information potential differences and |
| | molecular bodies throughout the biological system. |
| 3. | The current flow in the neuronal pathways (CNS, PNS, etc.) also can be affected by |
| | pulsed magnetic fields, which induce current flows, and comprises what is considered the |
| | primary informational system of the body. These systems act as LRC tank circuits. |
| 4. | The piezoelectric spike wave response in bone can induce current flow in the system |
| | and introduces crystal activated current flow informational components. |
| 5. | The diode action of the Purkinje cell processes in the hind brain and heart comprise a |
| | key organizing system in the human body's information channels. |
| 6. | Multiple coupled equations which describe the various informational and process |
| | channels of the human body. The coupling constant of the nonlinear terms of the coupled |
| | differential equations represents a recoherence term in which dispersive losses in the |
| | informational channels are overcome. This coupling term is expressible in terms of the |
| | soliton wave properties. |

V. Specific Biologically Active Frequencies and their Corresponding Systems of the Body Including Neuronal, ATP, Cell Potential, Hemodynamic, Piezoelectric and Endocrine System and Immune System Responses

A variety of neuronal processes are dominant informational channels of the human brain and body. Other informational channels exist, which control biochemical, psychological and other processes. We have designed the device and processes of this invention to properly represent both the time domain and the frequency domain information to properly represent that of a normally properly functioning human body. When applied to the body these correcting signals override, rectify, and re-modify and correct the bodies improper signaling so pain is reduced and health restored. See FIG. 10.

As is known in the state of the art nerves subdivide into branches and these communicate with branches of a neighboring nerve and communication between two or more nerves form a plexus. A plexus can be formed from the primary branches of the trunks of the nerves such as the cervical, brachial, lumbar and sacral plexuses and also the terminal funiculi as in the plexuses formed at the periphery of the body. Nerves divide, then join and then again subdivide in such a manner that each branch leaving plexus may contain filaments from each of the primary nervous trunk which from which it is formed. There is a free interchange of the funiculi and primitive fibers.

It is important to note that all individual filaments remain separate and distinct so that neuronal interchange of information occurs at the synaptic cleft in a digital manner. However, analog signal is also possible through electromagnetic Fourier informational signaling along the extent of neuronal pathways. The sympathetic nerves are constructed in the same manner as the cerebrospinal nerves but consist mainly of non-myelinated fibers collected into funiculi and enclosed in a sheath of connective tissue.

The sensory nerves or afferent nerves transmit information received by the peripheral extremities of the nerves and in this manner receives through the senses processed in the brain, external information to the brain. Also signaling from external sources can be received and utilized extrasensorally through electromagnetic and magnetic highly specific pulsatile fields. External entrainment fields operate though the sensorial nervous system and other excitable and responsive tissues. The motor nervous centers can either be excited by these external stimuli and excite muscular contraction or influence the process of nutrition utilization, growth, secretions and healing. Signaling can be to brain center by efferent signaling. Afferent and efferent signaling operates at about 8 to 10 Hz, which is similar to the 7.6 Hz signal emitted by the device of this invention and to the alpha brain power spectrum peaking. Nerve fibers terminate peripherally in various ways such as through sensory and motor nerves. Sensory nerves terminate either in minute primitive fibrillae or in special terminal organs which are peripheral end organs of several varieties one being tactile corpuscles which give rise to sensations. Also termination can occur in neuromuscular spindles which allow coupling to muscular, tendons and piezoelectric bone coupling effects. We utilize these processes in the design and operation of the device of this invention. Termination can also occur in the end bulb of Krause or modulated nerve fiber and corpuscles. This fundamental property of neuronal processes allows the inter-processing connection between external input and the internal information recording, transmission and connection to internal biologic tissues, organelles, organ, and organ systems of the human body.

For typical neuronal rates of 300 to 400 cm/sec and a minimal distance travel of 10-4 cm to up to meters in length, yield a time constant of 10⁻⁷ sec to 0.13 sec. The base frequency for these spectra of frequencies operates in a narrow range of around 7.6 Hz and for our purposes this frequency should lie within about ±0.05 Hz. This frequency is associated with the approximately standing wave hemodynamic wave of the iliac bifurcation, determined through measurements of mechanical oscillations.

In standard engineering practice, a low frequency can be used to modulate a higher frequency. We have found that, by utilizing the new principles of the device of this invention, we can modulate a low frequency with a higher frequency signal. This allows us to design a system which carries a greater amount of information. This informational system is designed to reflect the process utilized in biological signaling and hence the use of the device optimized human functioning. As has been demonstrated by the device and procedures for this invention a lower intensity higher frequency signal can ride on a greater intensity higher amplitude lower frequency. In this case a 70.25 Hz signal rides on a lower frequency 7.6/4=1.9 Hz signal and 2×1.9=3.8 Hz, the sleep frequency. One to 1.23 is the average pulse rate of the cardiac contractile cycle and 1.9 is a little less than double that value. The first signal is a square wave, and the second signal is a triangle wave. The third figure a higher frequency mix of 71.25 and 3040 Hz ride on a 0.04 Hz signal for proof of concept. Note that a factor of ten is 0.4 Hz which is a sub-harmonic of the 7.6 Hz signal.

The band width of 30.4 to 31.4 Hz of frequencies for the cardiac system determined experimentally and theoretically, which has a base frequency of 7.6 Hz. The fundamental base frequency for a square wave generated set of harmonics utilized in the device of this invention utilizes the half band width of 30.4 as 15.2 so that 2π/15.2=0.41 Hz. This is the base frequency of primarily all hormones. The fourth harmonic of this frequency is 1.6 Hz, which experimentally is observed to vary from 1.57 to 1.6 Hz. This slight difference is due to the nonlinearities of biological signaling processing, which is what we have measured. Note that 1.57=π/2 to first order. The fourth sub-harmonic of 31.4 is 7.85 Hz and that of 30.4 is 7.6 Hz. The actual signal frequency is 7.6 for the electrical conduction system due to the nonlinearities in biologic tissue processing. In vivo and in vitro Purkinje cell measurements for normally functioning cardiac endocardium and cerebellum Purkinje processes yield the 7.6 Hz signal. This is also the approximately standing wave frequency of the blood in the iliac bifurcation. We utilize this frequency as the viable and useful frequency for the device of this invention. Using this frequency as our fundamental frequency of the frequency spectrum of the device of this invention insures that the cardiac system remains in corrected biologic resonance. The lowest harmonics of 0.41, 0.82, 1.56, 3.2 are not necessary to be utilized but can be. The 7.6 Hz frequency is about the sixth harmonic of the adult average heart rate for males (72 bpm) and females (76 bpm) or a total average of 74 bpm or 1.23 Hz. This is for an undamped ringing square wave which has both even and odd components. Calcium ions and neuronal activity and bone production can be influenced by the second harmonic of 15.2 Hz which, when properly modulated by a higher frequency of 7.2 KHz controls and regulates Ca++ flow.

The following frequency values have been derived from what has been experimentally verified in nature for optimum information processing of the human body. This yields a frequency spectrum of 3.8 Hz, 7.6 Hz, 9.41 Hz, 70.25 Hz, 210 Hz, 352 Hz, 492 Hz, 647 Hz, 3040 Hz, and 7200 Hz. The associated bandwidths for these frequencies have been found to be 15.2 Hz, 30.4 Hz, 37.64 Hz, 281 Hz, 840 Hz, 1408 Hz, 1968 Hz,

2589 Hz and 12,160 Hz respectively for the above frequencies. These bandwidths are found to be approximately a factor of about four times the base frequency which comes out of the Fourier Bessel equations. We calculate and have measured the associated time constants.

For example, the 7.6 Hz frequency has a time constant of T=131.6 m sec. and τ=T/2=65.8 m sec (for a 50% duty cycle) fora bandwidth f_{BW}=2/π=30.4 Hz. The 70.25 Hz frequency, T=14.2 m sec and τ=7.12 m sec for f_{BW}=281 Hz. For the 647 Hz frequency T=3.6 m sec and τ=1.8 m sec for f_{BW}=2.6 kHz. For the 3040 Hz frequency, T=33 m sec and τ=165 m sec for f_{BW}=12.2 kHz.

Biologic tissue allows some variation which is necessary for responding to internally and externally occurring variability of conditions. Fundamental to biologic tissue is that it cannot be completely rigid. The device of this invention utilize this fact by the use of a dynamic intermix of frequencies to mimic normally functioning biologic material. The frequency specifications in the following table 2 are given for the device of this invention. Associated bandwidths and duty cycles and wave forms are given as well as the primary biologic system that utilizes these frequencies and to which the device of this invention is used to normalize.

In Table 3, we present the effective frequencies and tolerances used in the device of this invention derived from in vivo and in vitro experimentation of the fundamental resonances of the body's informational system. Bandwidths are found to be about a factor of four of their fundamental base frequencies. Duty cycles and wave forms are also given as well as the external field intensity at the skin surface to activate the associated biologic system which is also listed. Some attenuation occurs as the field output of this invention penetrates deeply into tissues. Proper design features are constructed so as the magnetic field strength induces the proper current and voltages at the proper site for correct tissue response and normalization. Because high frequencies can ride on and modulate lower frequencies, it is possible that such signaling processing can carry sufficient information content in order to effect and normalize and correct biologic processing in order to eliminate pain and enhance health.

The main treatment frequencies which are utilized by the device of this invention are: 7.6±0.05 Hz which corresponds to the cardiac Purkinje cell and hemodynamic iliac bifurcation processes, 70.25±0.5 Hz (primarily for use for females); 70.25 to 71.25+0.5 Hz (primarily for use of males) for the processes of the central nervous system CNS; 647±5 Hz for the processes of the peripheral nervous system (PNS) and other nervous tissues; 3040±10 Hz, related to the piezoelectric activity of collagenous material such as bone, muscle tendons and ligaments. These are the individual settings of each frequency. When the device of this invention operates, the intermix of frequencies produce a modulatory effect on each other in such a manner as to produce a frequency variation. For the 7.6 Hz emitted pulsed magnetic field, this yields: 7.6±0.5 Hz, 70.25±2 Hz, 647 to 680±5 Hz and 3040±10 Hz variations or greater. The abdominal aorta is divided into the two common iliac arteries at the iliac bifurcation which hemodynamically resonates at 7.6 Hz.

TABLE 3

| Frequency and | Band | Duty | Dominate | Intensity | Primary Biologic |
|---|---|---|---|---|---|
| Tolerance (Hz) | Width (Hz) | Cycle (%) | Wave Form | (G) | System and Effects |
| 3.2 + .6 | | | Triangle or | .1 mG to 2 G | Relaxation and Sleep |
| 3.2 − .1 | 12.8 | 25-50 | Square | | |
| 7.6 ± .02 | 30.4 | 50 | Square | 1.5 G to 3 G | Cerebellum, Cardiac, |
| | | | | | and Iliac Bifurcation, |
| | | | | | Hemodynamics |
| 9.41 ± .03 | 37.6 | 25-33 | Square | 1 uG to 1 mG | Cerebrum, Alpha |
| | | | | | wave enhancement |
| | | | | | (Improves brain |
| | | | | | function and reduces |
| | | | | | depression) |
| 70.25 ± .5 | 281 | 25 | Square | 5-50 G | CNS, PNS and Other |
| For females | | | | | Nervous Tissues |
| 71.25 ± .5 | | | | | |
| For males | | | | | |
| 647 ± 5 | 2589 | 33-50 | Pulsed Square | .1 mG to .1 G | Cerebrum |
| | | | and Triangle | | |
| | | | Intermix | | |
| 3040 ± 10 | 12,160 | 33 | Triangle Ramp | 5-50 G | Bone, Connective |
| (Based on ½ | | | Wave | | Tissues and Neuronal |
| wave value) | | | | | Processes |
| Full Wave | 24,320-28,800 | 33 | Triangle Ramp | 5-50 G | Bone and Collagen, |
| Value 6080- | | | Wave | | Tendons and |
| 7200 | | | | | Ligaments |

This swing in frequencies due to the intermix modulation matches the corresponding intermix of the biologic tissue activity. The frequency progression are related in a nonlinear manner but each frequency corresponds to an approximately factor of ten of the next lowest frequency. Signal accuracy or fidelity is thus maintained within the biologic tissue and these tissues correct information processing relative to each other. The requirement for corrective signal processing produced by the device of this invention requires the 3040 Hz upper limit of signal emission, which is read by the human body. This signal is approximately one half of the bone resonant frequency of about 7200 Hz at the upper end. Note that 2×3040=6080 but due to the nonlinearities inherent in biologic processing which requires somewhat less than a tenfold increase for fidelity of signal detection, reproduction, and transmission. The frequency of 7200 Hz corresponds to the piezoelectric response of bone tissue in vivo but is more like 6080 Hz in vitro. High frequencies can be utilized but are not necessary for the device of this invention. It is necessary only to utilize the 3040 Hz signal emission from the device of this invention in order to produce the required effect for pain reduction. In fact in vivo experiments have demonstrated the enhanced effect of utilizing the 3040 Hz emitted signal rather than the 7200 Hz response signal of the relevant to the piezoelectric tissue activity. Experimental tests in vivo verify the ethicacy of the use of the 3040 Hz signal frequency.

The intensity necessary for effecting the brain is much less than that for the CNS, PNS and other active tissues and the intermix of the 3.8, 7.6, 9.41, 70.5, 71.25, and 3040 supplies sufficient signaling for the brain. For most applications the 647 to 680 Hz frequency range is not necessary to utilize as the other systems require much more intensity but this frequency as a pulsed ringing square wave and/or ramp wave which is emitted from a probe having a cross coil orthogonal configuration which can be utilized.

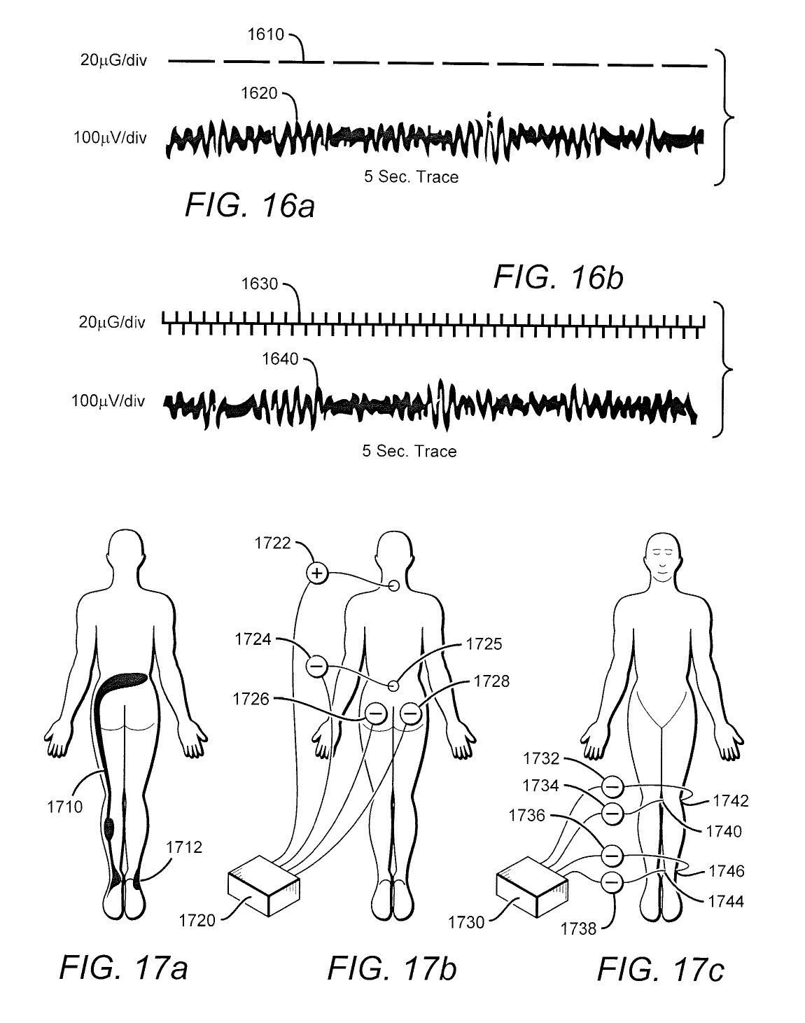

In FIG. 16a the top trace is of a no generated signal or control. The bottom trace in that figure is of a 31 year old female's Alpha EEG of 11.2 Hz. The trace speed is 5 seconds from end to end. In FIG. 16b, a generated signal of 9.4 Hz. is displayed in the top trace. In the bottom trace of 16b is the same subject Alpha frequency of 9.41 Hz. which is phase locked with the generated signal.

The method and operation of the device of this invention acts the same as certain specific environmental signals that are detected, and which are read in and corrected to the biologic system. The 9.41 Hz signaling is associated with the entrainment to the alpha brain wave state which is associated with relaxation and contemplation and act in analogy to the raster on the old analog TV sets. This state of consciousness is highly significant to relaxation, reflection and the foundational frequency range of the proper normal brain operation.

A. Piezoelectric Response and Bone

We have determined through in vitro and in vivo experimentation that electrical stimulation of bone produces a resonance in the kilohertz range. For the upper part of an adult femur, the resonant frequency is found to be about 7,200 Hz. Both dielectric and piezoelectric properties of bone are critically dependent on frequency. We have also determined that the bone structures of the body are resonant at 7,200 Hz and that collagen and cartilage also exhibit piezoelectric properties. Electrical output produced by applying stress on a solid asymmetric material produces a piezoelectric effect. In piezoelectric materials, stress and strain are coupled to electrical fields and polarization. The electric field vector is related to the piezoelectric tensor modulus, dielectric tensor, and stress and strain at relatively constant body temperature. The main subunit of compact or cortical bone is the osteon, a long narrow cylinder with a central canal that contains blood vessels and a nerve fiber which line up along the long axis of the bone as stiff reinforcing fibers. Collagen and hydroxyapatite crystals form concentric lamellar structures each about 5 microns thick. We derive our upper range frequency of 3040 Hz from our nonlinear resonance of tissue piezoelectric response and determined that we can utilize this frequency equally well or better than the full frequency range of 7,200 Hz to activate the positive piezoelectric response of the relevant body tissue for pain reduction. Both dielectric and piezoelectric properties of bone depend strongly upon specific frequencies of operation and also from extremely properly tuned sources of frequencies such as by the device of this invention. Reduction of osteoporosis may be affected by the device of this invention.

Collagen is the most common component of connective tissue and is piezoelectric and acts as a biological transducer connecting mechanical information into electrical information and is one of the biologic processes that allow the device of this invention to reduce pain. The inverse process is significant in the operation of this device, that is electrical signaling induced from the pulsed external fields of this device produce such signaling, which in turn is transduced into mechanical information in collagenous connective tissue and in bone. The piezoelectric materials of actin and myosin and other such biologic materials energetically regulate the microcosm of the cell. Thus the correctly imposed magnetic signaling can be imposed in such a manner as to correct signaling at cell levels to correct biologic functioning and, hence when so functioning, reduce pain and produce other benefits. The mucopolysaccharide portion of the connective tissue serves as a biological effector and is intimately associated with collagenous electrical response, which can be induced within the system or from externally imposed magnetic fields in order to properly regulate biological processes. Piezoelectric stimulation is a high potential extremely low current process. The body's piezoelectric response is one of many informational channels of the human body which involves electrical, mechanical, biochemical and hemodynamic processes. Piezoelectricity is one of many such mechanisms of the human body.

B. The Action and Properties of Nervous Tissue

The biologic informational and communications processing in the human body is briefly described herein to support and elucidate the purpose and methods for the specific design features utilized and which are profoundly effective in the device of this invention. Some of the systems and their pathways which are affected and enhanced by the device of this invention are as follows. The pulsed magnetic fields of this device and system can normalize such functions in the neuronal processes described herein. Some of the nervous tissues affected and normalized by the device of this invention are enumerated here. Then central nervous system (CNS) consists of the brain and spinal cord. The gray matter of the brain contains the cell bodies of neurons and the white matter contains the nerve axons and dendritic processes of these neurons. It is through the nervous system, in part, that the device of this invention operates and effects pain relief and elimination.

The peripheral nervous system (PNS) is that portion of the nervous system outside the CNS and is comprised of the 12 pairs of cranial nerves, which has a positive DC bias and the 31 pairs of spinal nerves. These nerves contain sensory and somatic motor fibers of the autonomic nervous system and comprise a region where the DC bias is negative for which the device of this invention is used to accommodate. The autonomic nervous system (ANS) is self regulating and functions independently and is the part of the nervous system that controls involuntary bodily functions and responds to changes in somatic activity of the body. The ANS operates to effect visceral effects and smooth muscle tissues. Treatment with the device of this invention reduces muscle spasms caused by pain and reduces the pain level.

The sympathetic nervous system (SNS) is the thoracolumbar division of the autonomic nervous system. Pre-ganglionic fibers originate in the thoracic and lumbar segments of the spinal cord and synapse with postganglionic nerves in the sympathetic ganglia. The post ganglionic fibers extend to the organs involved and effect skeletal muscles. Also regulated are the secretion of epinephrine and norepinephrine by the adrenal medulla, reducing stress symptoms can be affected by the device of this invention. Sympathetic effects are general throughout the body rather than specific to a localized region.

The parasympathetic nervous system is the craniosacral division of the autonomic nervous system. Pre-ganglionic fibers originate from nuclei in the mid brain, medulla and sacral portion of the spinal cord. They pass through the third, seventh, ninth and tenth cranial nerves below where the positive reference electrode coil is applied. They also pass through the second, third and fourth sacral nerves and synapse with past ganglionic neurons located in the autonomic terminal ganglia where the negative electrode coils are applied. Gland secretion is effected by the parasympathetic nervous system. Facial nerves can also be affected by the device of this invention to reduce pain such as dental pain.

Frequencies utilized by the device of this invention can effect brain neuronal and endocrine processes because these fields break the blood brain barrier, thus they are able to effect, in a positive manner, the neuronal and other processes of the brain to reduce and eliminate pain. The methods, procedures and device of this invention act in such a manner as to normalize and enhance the operations of the biological systems functioning. The CNS, PNS, ANS, SNS and parasympathetic nervous tissues are affected and normalized by the use of the device of this invention.

Neuronal tissues correct signaling depends on self entrainment or feedback cycle inherent in biologic process. We know that neuronal processes are directly affected by internal and externally generated pulsed electromagnetic fields. Other non-neuronal tissues have been found to respond to electromagnetic, electric and magnetic fields of precise parameters such as bone, heart, pancreas, lymphocytes, adrenal cortex and other tissues. Effects produced in the cerebral cortex can effect dopamine production and other neurotransmitter concentrations.

C. Cell Potentials and Charge Bias for the Life Battery Processes

The mechanism of neurotransmitter release is not well understood but there is strong evidence that suggests that there is a calcium dependent multifunction enzyme reaction involved. The calcium calmodulin dependent protein kinase (Ca⁺⁺, CaM-Kinase II) occupies a role in regulating the neurotransmitter release process. This complex enzymatic reaction is probably not directly involved in the triggering of the release of the neurotransmitter but it determines the amount of release that occurs with each action potential the significance of this process is known to those versed in the state of the art that calcium and calmodulin dependent protein kinase are very sensitive to the effects of ELF fields in the range of 7 to 20 Hz. Some frequencies in this range can enhance neurotransmitters in this region by effecting calmodulin. (Other frequencies in the 15 to 18 Hz range diminish its effects, which we do not use.) Hence, increase neurotransmitter production can be effected to reduce pain such as serotonin, endorphins and enkephalins.

The neuronal and other cell “life force” battery operates on a potential bias. In the case of bioelectric nerve potentials, the Na⁺ ion is of low concentration inside the cell but is relatively high on the outside and the permeability of the membrane for Na⁺ is very small. The interior contains K⁺ and Cl⁻ leading to the axoplasm having a resting potential of about −60 mV with respect to the surrounding liquid. This typical non-equilibrium condition is maintained by a continuous supply of energy in which Na⁺ ions diffuse into the nerve's interior and are transported back out. This system comprises one element of the human as an energy battery power source from the “sodium pump” powered by ATP.” The metabolic energy produced by ATP is primarily derived from oxidative phosphorylation of the respiratory chain as an analogy to the fuel (in biology food plus O₂) in an internal combustion engine giving off a CO₂ byproduct. The body or animal based biologic systems operate as an internal combustion engine taking in O₂, running an ATP metabolic process, eliminating CO₂ via the Krebs cycle. This complex system acts as an electrical, chemical and mechanical motor-generator using complex signaling to keep it in balance and operating correctly. The device of this invention couples electromagnetically to the biologic system to correct, enhance and normalize the operation of said system. Control and operational signaling can be applied both internally and externally. Hence the device of this invention becomes an adjunct of the human body. The action potential, which lets Na⁺ into the cell, is +40 mV. The change in influx of Na⁺ ions is associated with a change of permeability which lasts a few milliseconds. Waves of excitation propagate along the neuron as Na⁺ inflows and K⁺ outflows and travels to the synapse or end of the plate of the current carrier then active substances or neurotransmitters are released. The end of the permeability cycle occurs when acetylcholine is split into choline and acetate. (Neurotransmitters are stored at the synaptic site insides little sacs or synaptic vesicles.) At the end plate synaptic fibers release neurotransmitters. These are adrenergic and cholinergic nerves depending on the neurotransmitter active agent released. Adrenergic prostaglandin fibers release nor epinephrine as an “accelerating substance” which is a fast acting excitatory neurotransmitter of the CNS. At the motor end plate, many synapses in cholinergic nerves release acetylcholine giving rise to rapid inhibitory action. Both inhibitory and excitatory action are ascribed to dopamine which replaces nor epinephrine and serotonin. All these processes are acted upon and normalized over a long duration which lasts for months or years by the method, device and system of this invention, when using one or a series of properly applied treatments.

The basic structure of biologic tissue, the cell, carries a resting and active cell bias due to the various levels of cell membrane permeability of K⁺, Na⁺, Ca⁺⁺ and Cl⁻ acting as ion transport and exchange channels. The cell membrane acts as an in parallel RC circuit. This is true of all cell membranes which are lipoproteins. These tissue elements carry this potential voltage bias whether they are non-excitable (non-neuronal) or excitable (neuronal) tissues. The bias between the intracellular and extracellular potential in some example cells as skeletal is −96 mV, muscle −50 mV, or neuronal −73 mV. There are 210 cell type structures in the human body which all carry a RC net work like voltage bias. These bias voltages always exist in living tissues. The impedance of the cell membrane is high and on the order of 10⁸Ω the energy of ion transport across the cell membrane is accomplished through the Ca⁺⁺ pump which uses up one ATP molecule. In the case of the single neuron in the Giant Squid in the HH model, the system acts by Ohm's Law in a linear manner. Excitable tissues can be modeled after LRC tank circuits which mimic the proper nonlinearities in the circuit. In fact, all interconnected tissues of the biologic systems operate nonlinearly--that is electronically as a DC biased AC resonant low loss circuit. Low ion concentrations can have a large voltage charge in this high impedance system.

The various ion transport characteristics of the ion subset K⁴, Na⁺, Cl⁻ and Ca⁺⁺ is what gives rise to cell resting potential in the plasma membrane. Depolarization and repolarization of the cell membrane in excitable tissue allows information to be impressed along the current flow pathways. In cell membranes, voltage gated channels have various electrical characteristics. For example, from small to large channel size, the four unit class is a voltage gate for K⁺ and Ca⁺⁺ ions. The five unit class is Ligand-gated and the sub unit acts as a (semi conductor) gap junction for all ion channels. Ion one-way gating has the specific properties of a semi conductor and neurons propagate in an isolated conductor from an initiated action potential that propagates to the end of the axon. Neuronal responses and cellular responses are of such kinds as excitation, inhibition, facilitation, and cycle summation or threshold. These current transmission or current inhibition diodic threshold “and” logic gates are excellent electrical component analogies to biophysical processes. The systems of the body as electrical components, informational systems and logic gates as does the device of this invention hence it can facilitate healing, biologic process normalization and pain reduction with the method and device of this invention.

Electrical activity is fundamental to life. The electrical activity of nerve cells and, indeed, all cells, depend on the movement of charge, carried by small inorganic ions across the plasma membrane. Faraday's law directly relates current flow to magnetic fields. Protoplasm is the class of complex nitrogenous compounds which occurs naturally in animals and plants and yield amino acids when hydrolyzed. Amino acids contain proteins which are essential for the repair and growth of animal tissue. The transmembrane ion flow gives rise to the membrane potentials, the firing of action potentials and their grouping in complex temporal patterns. External stimuli can create changes in the transmembrane ion flow. The key question is the manner in which the ions appear to move across the plasma membrane. The lipid bi-layer of the plasma membrane is a very good electrical insulator so that charged species are largely unable to penetrate the membrane without utilizing a great deal of energy such as in a frequency variation mode. One of the mechanisms believed to operate to drive the ions across the barrier is by an energy driven “pump” or carriers which use the energy from ATP hydrolysis, it is currently believed, that this processes can produce active transport processes to transport ions against a concentration gradient. The induction process reduced by magnetic fields effects the manner in which ions can propagate across such a high impedance barrier. To affect many cells and all electrical signaling in most nerve cells.

Enzymatic reactions involving protein kinases occur when the terminal phosphate group from ATP altering the electrical potential on the phosphate group which is negatively charged PO₄⁻³ tertiary phosphate group. The ATP hydrolysis mechanism acts as an active transport mechanism against a concentration gradient which acts as a central gating like a diode. It has been observed in vivo experiments that in cases where metabolic processes are too slow, their increase can be produced by external pulsed magnetic fields such as those produced by the device of this invention activate neuro tendon response has been observed as well as effects such as lactic acid production in muscles and expulsion into the blood stream. Muscle stimulation by electromagnetic and magnetic fields of the device of this invention and bone piezoelectric stimulation have beneficial effects on health.

The sodium-potassium pump involving the ions Na⁺, K⁺, Cl⁻, Ca⁺⁺ and other cellular properties including the insulative lipoprotein cell membrane, leads to the approximately −70 mV potential between the negative cell interior and positive charge outside the cell membrane. This differential holds charge like a capacitor, but does not fully discharge and acts as a charge generating system. The energy system of the body is ATP metabolism which uses the Ca⁺⁺ ion and acts as a distributed battery throughout the system as a whole of the body. This is the human battery power system of the body at the physical level. The biological battery is analogous to a series plate capacitor which is fluid filled. Not only does the cell membrane act as a container for cytoplasm and protoplasm but has variable permeability to on exchange and hence act as a control gating grid. The cell membrane also acts as an insulative layer to maintain the charge bias for the biologic battery effect. Biologic insulation is supplied by cell membrane lipoprotein and neuronal myelin sheathing. Each of the ten trillion cells of the human body holds charge in the manner of a capacitor. This capacitance varies due to the change in permeability of the cell membrane. The supply voltage depends on its exchange of Na⁺ and K⁺, ions across the barrier. The electrical energy supply is produced by the ATP process that produces battery energy as a near continuous supply of a trickle charge. The approximately three pound brain can generate 20 watts and the body up to about 50 to 100 watts.

The cell membrane is comprised of lipoproteins. To sense their environment, cells rely on their receptor proteins that permeate their surface. The receptors latch onto specific molecules which trigger a cascade of biochemical events that lead to specific cell processes such as the secretion of hormones or pathogen destruction. In the case of mast cells, cell membrane protein receptors, when activated produce histamine. Before this occurs, a spike in the intercellular calcium occurs.

Chemical potential in which bio energy resides, before it is transformed into mechanical energy and other useful energy forms the ATP. For example, the resting ATP at the cell muscle is about 5×10⁻⁶ mole/gram of muscle is sufficient energy to contract the muscle in less than one second. As a series of timing mechanisms occur throughout the body. In addition to some already mentioned are the 30 Hz nystagmus of the eyes (similar to 30.4 of the cardiac band width (and the 10 Hz efferent—afferent neuronal feedback firing to control muscular motor control (which is near the alpha power spectrum peaking). At the high frequencies is the resonant frequency of the cell membrane lipo protein at 100 Gigahertz. Also of course is the visual perception at 10¹⁵ Hz and infrared (IR) perception by the skin and perhaps pineal at 10¹⁴ with a wave length of cell size. Melatonin skin production responds to ultra violet (UV) A and B (for release and production respectively) at around 1017 Hz as well as producing the necessary Vitamin D. It has been noted that certain specific RF frequencies are absorbed by the human body. The body operates on a series of frequencies and frequency harmonics as its fundamental mechanism and that is the reason why it responds to the properly imposed external frequencies.

Although there is no distinct specific pain sensing tissues that have been identified as unique to the sensation of pain alone, a number of systems of the human body receive and transmit information. The whole of the biologic system is involved with pain perception and response to that perception. In chronic pain, a feedback loop occurs in which pain sensorial perception and response produces a “run away” effect, further aggravating the system producing negative responses in the system causing lost information which affects the biologic system as a whole.

D. Endocrine Hormonal Response to Electromagnetic and Magnetic Fields

The thalamus of the brain is one of the key organs for processing and transmission of information. The hypothalamus processes information and leads to the response of stresses such as fear, thirst and other sensations which allow the system to adjust to external and internally generated information. Pain is one such stress, which through the hypothalamus affect the whole system and associated emotional states. By pain elimination, the hypothalamus generates less stressful responses which also affect the endocrine system in a positive manner. The hippocampus regulates and stores memory and is part of the memory cycle of pain perception that continues to produce information in the system as a whole that continues the information refreshing cycles that lack the proper Fourier component carrying informational components. By emitting an externally generated signal that through entrainment is read by the body as its own, it connects to this signal because of the restorative properties of biological systems to optimum functioning. The pons and the cerebellum act in a similar functional manner to the hypothalamus for the cerebrum. The hypothalamus controls the network of the endocrine gland system, which secrete the twelve major hormones of the human body. A gland is an organ that separates certain specific biological fluid from the blood and glands secretes these elements into the blood stream in two manners. Then it secretes these fluids in a timed manner into the blood stream or lymphatic system.

The active principles or chemicals produced by these glands are called hormones and effect tissues more or less remote from their place of origin. The endocrine system produces homeostatic control utilizing its feedback system to control the proper levels of the various hormones. For example, the parathyroid gland regulates the level of blood calcium and hence controls the overall level of excitability of the sodium-potassium neuronal pump and hence neuronal activity. Duct glands empty these elements into an organ such as the liver and kidney and then into the blood stream and ductless glands which pass secretion directly into the blood system. The hormones of the endocrine system are directly associated with the perception of pain or the absence of pain. If proper electromagnetic signaling occurs, then proper serotonin, endorphins, enkephalins, and dopamine levels are maintained such as by the use of the device and procedures of this invention, thus eliminating pain and restoring proper biologic functioning of the human body.

Specific frequencies of radiation affect the low frequency and extremely low frequency endocrine and also the hormonal system, particularly through the action of catecholamines. These fields have specific effects on acetylcholine, which controls memory processes, motor control, and physiological variations in circadian cycles. Disruptions of the cholinergic system, produced by various abnormalities can give rise to some of the symptoms of discomfort, sensation of irritation and pain. The autonomic system functions through the action of medulla oblongata and pons controls sleep, appetite, heartbeat, body temperature, and other autonomic functions. Autonomic and parasympathetic systems are very sensitive to externally applied ELF, and higher frequency emanations of very specific wave forms, frequencies, duty cycles intensities processes related to the limbic system or hypothalamic-pituitary axis and the ganglia involved responses so that the interaction of the limbic and pons systems which indicate a possible positive ELF activity effects on emotional states. Of course, reduction and elimination of pain are associated with positive emotional states. Positive states are directly achieved through positive effects of pulsed magnetic fields on the autonomic nervous system.

Increase levels of the “stress related hormones” of cortisol and adrenaline, which is associated with the reported sensation of pain. We can measure the levels of stress related hormones as well as endorphins and enkephalin levels and serotonin levels associated with relaxation associated with reduced pain level before, during and after pain treatment with the device of this invention. Also blood volume, particularly to the limbs, has been measured and has been shown to increase during and after the use of the pain reduction and elimination system and device of this invention, indicating additional enhanced effects. We use these parameters and the pain objectification part of the device of this invention as an indication of the effectiveness of pain treatment and enhanced biologic functioning. These can be measured in vivo before, during and after treatment with the device treatment and during controlled testing conducted where active and inactive device conditions which are used detached battery under double blind conditions. This procedure has allowed us to identify and objectify pain treatment effectiveness. The increased levels of endorphins, enkephalins and serotonin soon return to their proper normal levels for a healthy human in about 24 hours but the pain reduction and relief remain. If cortisol, noradrenaline and adrenaline were elevated or become out of balance under resting conditions before treatment, these hormone levels return to normal after treatment and remain at the approximate proper physiological level.

Endorphins are polypeptides which are produced in the brain that acts as the body's own natural pain reduction system. Endorphins produce their analgesic effect by binding to opiate receptor sites involved in pain perception. The pain threshold is therefore increased by this action and hence endorphin production and normalization by the device and procedures of this invention reduce and eliminate pain. The most active of these compounds is beta-endorphin. These measures are found to correspond to less sensitivity to the pain objectification device of this invention.

Enkephalin is a pentapeptide produced in the brain and also acts to produce analgesia by binding to opiate receptor sites involved in pain perception. By normalizing the enkephalin levels, which are often too low in chronic pain patients, the threshold of pain perception is therefore increased by the action produced by the application of the pain reduction device and methods of this invention. Prostaglandin is any of a group of autacoids, which are not hormones, having physiological actions such as fluid balance, platelet aggregation, blood flow and neurotransmitter function as well as anti-inflammatory response. Inflammation can produce pain and can be associated with increased histamine production. We have found that the use of the device of this invention reduces and can eliminate inflammation and hence reduce pain from such sources.

Catecholamines such as norepinephrine and dopamine are produced in the brain, the hypothalamus and also found in the cardiac system. Some of the catecholamines, which are associated with states of well being, are epinephrine, norepinephrine and dopamine. These biologically active amines, derived from amino acids and tyrosine, have a marked effect on the nervous and cardiovascular systems, metabolic rate and on smooth muscles. The neurotransmitters, which are associated with well being and reduced stress levels and reduced pain, are norepinephrine and serotonin which have the precursors of tyrosine and tryptophan, respectively. They transverse the synaptic cleft before they are broken down by the enzyme monoamine oxidase. It may be, through inhibiting the effect of monoamine oxidase, that the increase in noradrenaline and serotonin is affected and that their beneficial activity is enhanced and prolonged in the body. It is believed by those versed in the art that these neurotransmitters affect the hypothalamus and limbic system.

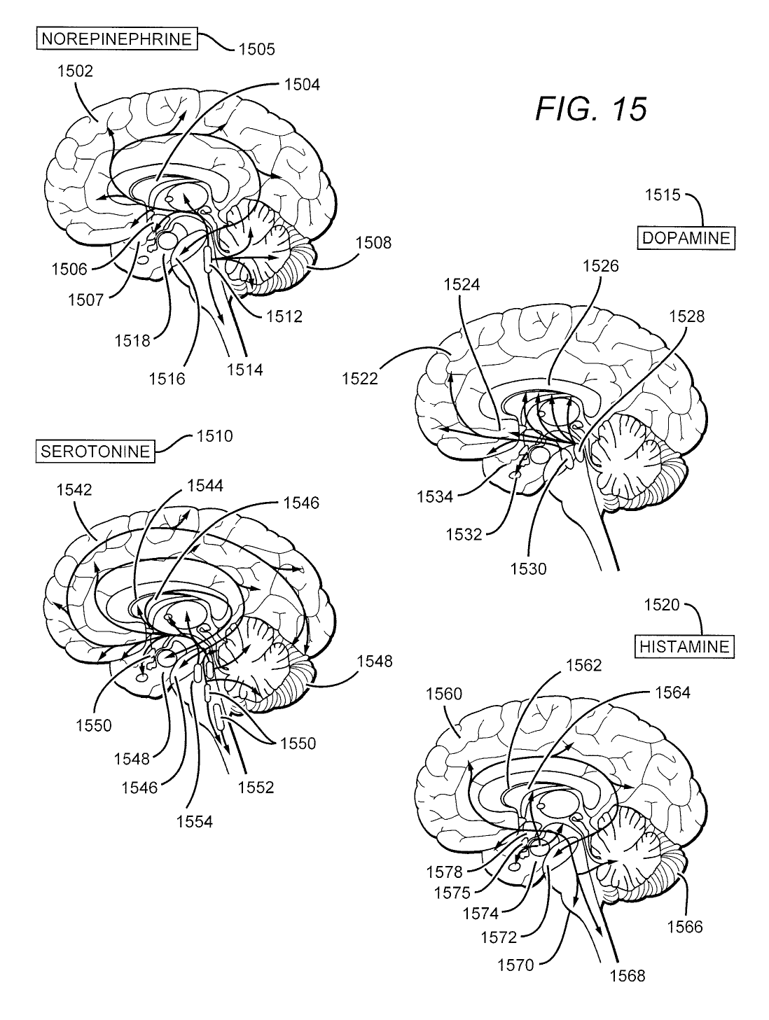

Norepinephrine and serotonin cannot break the blood-brain barrier between the body and the brain. The brain normally only received some of the many substances that circulating in the blood plasma through its own production of these substances. The brain remains in chemical isolation due to special structural features in the capillaries or blood vessels surrounding brain cells. These capillaries are constructed so as substances must directly pass through the cell membranes rather than through small clefts in the walls of the capillaries as occur throughout the rest of the body. Generally, substances pass into the brain in proportion to their ionic charge, size of the molecule and solubility with the lipid outer membrane shell of the capillaries. Properly pulsed magnetic fields are not restricted by the blood brain barrier and pass readily across it, carrying the proper chemicals and substances with them. Utilizing the correct and highly specific electromagnetic frequency signatures allows these fields to direct the proper biochemical passage of optimum pain reducing bio chemical to pass through the blood-brain barrier. When properly tuned, the device of this invention allows for the proper production and maintaining of neurotransmitters. It is clear to those versed in the state of the art that electromagnetic fields can transverse the blood-brain barrier. This fact can be used to augment proper neurotransmitter balance for reduction of and elimination of pain and maintaining proper stasis in the human body. Some of the neurotransmitter pathways are given in FIG. 15.

The proper formation of neurotransmitters is part of the process of degradation or breaking down of these molecules into simpler substances or end products. Norepinephrine breaks down into the compound MHPG, which is secreted in the urine. Serotonin is broken down into the product 5HIAA. The amount of these substances can be deduced by measuring MHPG and 5HIAA in the urine. Increases in these substances in the urine can be observed in the case of the use of the device of this invention and are formed at their correct biologic levels. People in pain or suffering from depression often display reduced levels of MHPG and 5HIAA. Also measured of these end products can be made by spinal fluid taps, although this invasive measure is not recommended. The proper balance of norepinephrine and serotonin, which effect the neocortex, thalamus, hippocampus and cerebellum primarily, appear to be correctly maintained over long periods of time after treatment with the device of this invention.

Other neurotransmitters such as acetylcholine affect the neocortex and hippocampus as well as the septum and stratum. Dopamine affects a number of regions of the brain such as the frontal lobes, septum, stratum, ventral segmental areas as well as the olfactory bulb but not the cerebellum. Pulsed magnetic fields are found to enhance the proper exchange process throughout the body and through the blood brain barrier, particularly small molecules such as sodium, potassium and calcium which can be driven to exchange across this barrier to reduce pain and enhance health.

The proper balance between serotonin, dopamine and the histamine levels must be maintained in the brain. It has been found in vivo and in vitro animal studies that these proper levels can be maintained through the use of the proper combination of external pulsed magnetic fields exposure. Neurotransmitter and mast cell assays were conducted in sacrificed animals (albino rats). When properly constructed pulsate magnetic fields were applied that corresponded to those emitted by the device of this invention, proper biochemical and physiological parameters were observed in the in vitro assay of brain slices in the cerebrum and cerebellum gray matter. Histamine produces the inflammatory response in order to produce local blood increased volume for healing. Too much histamine produces allergic responses and too low a histamine level relative to too much serotonin levels can cause shock. Rebalancing the ratio of these levels by properly affecting mast cells can overcome the extremes of imbalances of these substances in the brain as pulsed magnetic fields readily pass through the blood brain barrier. Serotonin acts as a potent vasoconstrictor and its proper levels of production in the brain and body which can be controlled by the device of this invention can be used to reduce the effects of trauma and associated shock.

The macromolecule of metabolism is adenosine triphosphate synthase (ATP) which is the universal energy mechanism of living organisms. It acts as a rotary motor and adds phosphate groups to molecules of adenosine diphosphate to produce ATP. As it rotates, it converts chemical energy to mechanical energy. This is performed as discrete shifts of the rotation motional of bursts of two primary frequencies of 0.8 to 10 Hz. This system is a molecular amplitude modulated system. Also the motor action molecules of myosin are what powers the process of muscles and also is an amplitude variation modulated signal. The associated frequencies for this system are about 2 to 10 Hz. Low end frequency emissions, properly timed from 0.41 Hz to 15.7 Hz can be utilized to affect these systems.

Serotonin or 5-hydroxy tryptamine (5-HT) is a neurotransmitter of the central nervous system (CNS). Serotonin is present in platelets and mast cells. Increased production of serotonin is associated with relaxation and well being. The mast cells of the brain do not circulate in the blood. They contain hydrolytic enzymes and serotonin. Mast cells are also present around blood vessels of the skin and bone marrow. We know that mast cell regulation can be effected or controlled by properly timed and proper wave form ELF fields up to approximately 10,000 Hz fields. See FIG. 15.

It is also through positive effects on the hormone levels and proper neuronal transmitter uptake levels that allow for long lasting positive effects of this invention. Since the effects produced by the device of this invention effect and produce naturally occurring corrections to the biological system, is the reason side effects do not occur. The nerve cell consists of a cell body and its processes which are an axon and one or more dendrites extending from the cell body. The axon transmits nerve impulses and the dendrites receive impulses and then transmit them to the cell body. Nerve endings or termination of a nerve fiber, an axon or dendrite, may be a sensory receptor or motor effector, repetitive refreshing of the system occurs due to afferent and efferent neuronal firings which occur at a signal rate of about 10 Hz. The eye nystagmus movement which refreshes visual perception occurs at about 30 Hz. Both these frequencies are intermix frequencies produced by the device of this invention. Note that the power spectrum peaking of the alpha brain refreshing faster is also around 10 Hz and varies from 7.6 to 14 Hz. The afferent-efferent approxithately 10 Hz neuronal processes comprise a significant feedback loop that needs to be properly maintained. Brain wave alpha entrainment, relaxation, and reduced depression occur with the use of the 9.41 Hz frequency and into the cerebrum.

E. Cell Potentials and Neuronal Electromagnetic Processes

Every cell maintains its soupy interior at a lower potential than the tissue or the blood surrounding it. The cell membrane potential is about −70 to −80 mV and maintains the proper potential by the maintenance of the correct concentrations of sodium, potassium and calcium and other ions. The device of this invention is designed to produce the proper intensity output so that when it is indirectly coupled and linked to soupy tissue, the impedance of this soupy tissue gives rise to the proper induced current flow.

The magnitude of typical transmembrane potential gradients is 10⁵ V/cm which indicates an amplification process which amplifies the effect of weak internal and external fields. Cellular systems act as large capacitive amplifiers due to the high transmembrane potentials. Capacitive coupling in high dielectric materials is a key to storage potential reservoir of biologic information. Specific external information can be stored and amplified through capacitive coupling. Proper placement of electrodes is designed and based on the above physiological and biochemical parameters.

The plasma membrane acts as a capacitor and resister connected in parallel, having a membrane time constant of T=RₘCₘ in which the voltage charge occurs exponentially with time, Vₜ=V₀ exp.(−t/T). Time constants can vary over a wide range even through Cₘ per unit membrane surface area is remarkably constant at about 1 μf/cm² in all membranes. The fixed capacitance impedance Xc allows the current and voltage values to vary in a proper manner. Control of current flow, and hence voltages, can be affected through externally applied magnetic fields.

Neuronal depolarization and repolarization involves a threshold effect, below which no response occurs. In general, for neuronal tissue the threshold depolarization Vᵣ varies from 10 to 20 mV. Above the threshold, large charges in membrane potentials occur in several milliseconds associated with an approximately lower limit of 1000 Hz signaling. This is approximate since biologic tissue involves a complex dispersion relation between tissue and frequency. In fact, experimentally we determine that this membrane potential change occurs with an associated frequency of the order of 3000 Hz. Other biologic tissues carry different resting potentials and have somewhat different capacitance and a wide variety of time constants. Fourier components from 7 to 8 Hz and 70-78 Hz signaling occur up into the Kilo Hertz region. Output frequencies from the modulation with the 3040 go up to above 50 kHz, and the higher their frequency, the lower their amplitude. Hence, this is the reason the device of this invention is so effective due to the large number of Fourier components and their associated effective time constants.

At the very high end of resonant frequency is the lipoprotein of the cell membrane at about 10¹¹ Hz. A great deal of work has been conducted on the DNA, t-RNA, microtubules, etc. The DC polarity in a single frequency or emitter device is due to the fact that one side goes to the battery terminal and the other side is connected to the ground terminal. A frequency difference is found in a single coil between its two opposite ends as we utilize an asymmetric pole piece. In a dual frequency emitter system, two beat frequencies relative to each other, the polarity is produced by a phase shift either clockwise or counterclockwise from the end of the coil pole piece due to the phase relationship of 7.6 Hz and 70.25 Hz or 71.25 Hz signals. No frequency difference between the two coil poles occurs.

In the case where we can utilize such as six approximately 600 coils in parallel, the load across the output of the pain reduction and elimination device of this invention, creates an approximately 100Ω load. For each of the 600Ω coils, we have about 2.1 Henrys of inductive reactance using a band width of 31.4 Hz related to the 7.6 Hz emitted signal where f_{BW} ∝1/RC. The induction is determined from the frequency resonance and capacitance of the AC circuits. For 53 μf (micro-farads) then

$$L = \frac{1}{2 π {f}_{0} {C}_{2}}$$

where f₀ is the frequency, and C₂ is the relevant capacitance of the circuits of the device of this invention. For the 70.25 emitted signal, a factor band width is about 300 Hz and for the associated capacitance, we have an effective inductance for the inductance of two coils in parallel

$${L}_{r} = \frac{{L}_{1} {L}_{2}}{{L}_{1} + {L}_{2}}$$

for two coils and the mutual inductance M=K\sqrt{L₁L₂} for K the coefficient of coupling, with a maximum value of unity. For a resonant AC circuit, we have

$${f}_{R} = \frac{1}{2 π \sqrt{LC}} .$$

The capacitance can be determined from an RC circuit in which

$$f = \frac{1}{RC}$$

then

$$C = \frac{1}{fR}$$

where for the base frequency of 7.6 Hz for a for the example use of 600 coil, R=600, then the effective capacitance is 4.6×10⁻² farads or 0.46 mf (micro-farads). We can now calculate the relevant inductance and resistive loads. Note that coils of other resistive and reactance can be utilized.

The device of this invention is based on both capacitive coupling and impedance coupling into the human body. This is accomplished through both inductive and capacitive coupling from pulsed magnetic fields producing current flows in soupy tissue. Pure capacitive coupling blocks DC and pure inductive coupling blocks AC. Biologic tissue is both DC and AC and is both capacitively and inductively coupled. The body must pass both DC and AC where appropriate and sometimes both through the same biologic process. The device of this invention accommodates and induces both capacitive and inductive coupling to induce proper tissue functioning.

Due to capacitive coupling after the proper frequencies are set in the time and frequency domains the total modulation swing allowed is greater than that in Table 3. Each is set by isolating that part of the circuit and then setting the proper frequency within that tolerance. When the device of this invention is in operation, capacitive coupling allows a greater variation of the frequencies involved which better couples to the active biologic tissue involved. Note that the observed variations in frequencies, when operating in concert, are inter-modulated, and yield a larger value than is given in Table 3.

To produce the proper frequency response and proper beat intermix frequencies, the correct timing and mixing of the three primary frequencies and their characteristic harmonics of the pain reduction device must be produced. Inductive coupling of the 7.6, 70.25 or 71.25 and 3040 Hz frequencies must be inductively coupled and tuned in such a manner so as to produce the proper signaling to induce the correct informational processing of the various systems of the body to reduce pain. Other embodiments of the device of this invention utilize the 1.57 Hz and 647 Hz signals and other signals as well.

In order to produce the proper signaling for the preferred embodiment, a 3040 Hz with about (±10 to ±40 Hz) triangle wave oscillator chip using about a 33% duty cycle is modulated by a 7.6 Hz (±0.5 Hz to ±5 Hz), about a 50% duty cycle pulse signal which is generated simultaneously with an approximately 70 Hz to 71 Hz (±0.5 Hz), at about 25% duty cycle pulse. More leniency of tolerances may be allowed in other embodiments of this invention. The 7.6 Hz plus the approximately 70 Hz pulse generator chip must be able to modulate the 3040 Hz generating chip circuits. The output of the three frequency generated intermix must be able to deliver approximately one ampere of current as a maximum into a one hundred ohm load for the preferred embodiment of this invention. The one hundred ohm load is comprised of at an optimum six parallel coupled coils or the equivalent in order to produce a moving or dynamic field of ten Gauss or 10, 20 and 30 Gauss emission in each coil or ceramic or other type of emitter to produce a proper pulsed magnetic field less than other output intensities such as 0.1 G to 55 G or more can be utilized. Closer tolerances are more useful, that is one can utilize about a 7.6±0.1 Hz and about a 70±1.5 Hz. More or less coils can be utilized in various embodiments of this invention. Frequencies are set separately but the intermix modulation allows greater frequency variation and hence the device of this invention is not limited to all the frequency constraints given in Table 3.

The about 7.6 and 70 Hz frequencies utilized are ringing square waves and the 3040 Hz signal is a forward leaning ramp or sawtooth wave, which can be considered as a modification of a square wave. The ringing square wave not only produces the odd harmonics but produces even harmonics of a lesser amplitude. This proper mix of frequencies, wave forms and intensities of the emitted signals from the pain reduction device of this invention are required for the proper functioning of the herein pain reduction device.

For a resistive load of R for the inductive impedance, Z_{L}=R+i2π∫_{BW} L where L=2 Henrys, for example for six 600Ω coils in parallel, R=100Ω. For the example for frequency of 7.6 Hz and f_{BW}=30.4 Hz and the current is about i=3 mA, then the Z_{L} impedance is 630 ohms at the site of the coils. For R=600Ω the inductive impedance Z_{L}=131 ohms. For R=100Ω, and for f=70.25 Hz, Z_{L}=216 ohms and for f=648 Hz, Z=1.08 k ohms and for f=3040 Hz, we have Z_{L}=5.04 k ohms. In general, for a good approximation, a 600Ω coil generates precisely 2.1 Henrys from measurement.

Each frequency is modified by the next lowest frequency in sequence to form the proper modulating feedback process in order to reduce and eliminate pain. Remote pulsed field electrodes from the device of this invention are placed over myofascial trigger points, sites of injury, locations of surgery and sites of pain objectification by the pain objectification system of the device of this invention. Cutaneous electrodes can also be used when deemed necessary, utilizing the connecting impulse fields of the device of this invention described herein. Proper diagnostic and complementary medical procedures are utilized.

F. Biological Gating, Cell Membrane Potentials

One of the major keys to the input-output electromagnetic system and the internal and external self interactive and environmental system (or outside) interaction is through a diode gating system within the biologic system. External fields are generated by the device of this invention to normalize natural processes within the biologic systems. Diodes like processes occur in biologic tissues and act as gating processes. This gating system detects and allows certain specific wave forms intensities and type of external fields to effect and interact with biologic tissues, activating detection gating processes which can occur naturally in the normal healthy body and can be entrained to maladaptive body processes and tissues.

Slow drift conductive electrons do not have the potential to break metal surface—if they have a certain minimum energy for different metals for electrons to escape. If the barrier energy for electron escape for that particular metal is supplied by photons or other energy electrons can escape from the surface, an isolated conductor becomes positively charged. The electrons form a space change of negative charge. If an earlier conductor has a higher, potential than the first, its electron cloud of charge is attracted to its second conductor and as long as the two conductor's relative potential is maintained, then there will be a steady drift from the emitter cathode to the plate or anode. This is the standard model of the original diode. Now solid state ICs are utilized, but a better analogy to biologic gating is the old electronic tube technology. Such an electron arrangement with electron and electron holes is semi conductors that give rise to a one way gating process. Similarly, ionic tissue or conductive wire with current flows do not “gate” but biologic tissue does. Impulse gating can occur, that is for a pulsed current flow or a permeate potential difference between two parts of a semi conductor like system can be maintained as a steady potential such as on the cell membrane. The same grid control applies in IC as in tube diode and triode technology. The crystal grid control is analogous to the charges in cell membrane tissue permeability. Cell potential surface voltages are driven in an analogous manner.

The human biological processes have many properties of electrical circuits and electronic components and operate in like manner. We present certain specific examples of the manner in which biologic tissues operate as electronic components and circuits. Key specific examples are presented but there are many others. Some of the major entry points into the body from externally generated fields for the device and method and internal processing of electromagnetic and magnetic fields are through the Purkinje process, myelinated and unmyelinated neuronal processes, induced magnetic response of the paramagnetic heme, piezoelectricity of the bone, dielectric properties of collagen, and cell membrane potential. These systems and other of the human body act as transmission lines, amplifying elements recohering processes, semi conductors, receiving and emitting antennas, rectifiers, insulators, charge and potential reservoirs, band pass system operating both in digital and analogous modes.

Various tissues of the biologic systems, cells, neurons, glial cells, lipoproteins and fat cells as well as myelin sheathing act as conductors, semi conductors, rectifiers, insulators and gating systems and have strong analogy to electronic components. Neurons transmit electric and electromagnetic signals, and magnetic signaling in the paramagnetism or induced magnetism of the heme. We know that externally generated magnetic fields, of course, induce current flows in any conductive tissues but the structure of these tissues may involve one way gating, feedback control, insulating behaviors and numerous other electronic control like mechanisms. A good analogy for a biologic rectifier system is a “cat whisker” crystal radio set which uses a conductive metal on a semi-conductor substrate (such as Galina, PbS Iron Pyrite, FeS and Germanium) and operates as a nonlinear gating junction. This nonlinear system behaves like diode. A varying AC signal influences current flow in a preferred direction, i.e., acting as a diode and uses a tunable LC network. (Schottky diodes are of particular interest in modeling biologic tissue processes and have high barrier rectifiers for conductive material (metal or ionic tissue) and a large band gap for a semi-conductor.) PN Schottky diodes are low current, high rectification, composed of minority carriers, and certain biologic processes operate in an analogous fashion to them. In biologic tissue, the analogy to a band gap requires an extremely low threshold of energy. See FIGS. 1, 4 and 10.

G. Results of Preliminary FDA Trials and Some Representative Example Clinical Trials

The FDA study demonstrated the efficiency of the device of this invention in reducing and eliminating pain with no negative side effects. The FDA study was primarily conducted on patients with back pain. A number of FDA and other clinical trials were conducted on other sources of pain such as chronic pain, current injury and surgical recovery. Some of the many treatment cases have been for orthopedic nature such as sprains, strains, hematoma, tendonitis, bursitis, arthritis, inflammatory muscle—tendon syndrome, also treated have been surgical recovery involving such as in soft tissue, orthopedic and other surgery, current soft tissue injury due to deep cuts and dental pain.

Single and double blind studies of well over 200 male and female patients from 21 to 95 years of age, have been conducted and analyzed with over a 89% statistical significance success rate per patient in pain reduction. When a full treatment regime has been conducted pain elimination has been at the 96% level. We present some typical examples. The field strengths at the multi pulse coil emitters was from 5 to 30 Gauss but is not restricted to these intensities with a series of highly specific frequencies in the range from 7 Hz to 4 kHz but not restricted to this range and can be of 0.4 Hz to 50,000 Hz or more. Multi frequency waves of about 30% to about 50% duty cycle were used. The single and double blind studies can be conducted and compared to control runs (with the device in a non-active mode). This is possible because this non-invasive method and system cannot be directly sensed by the patients, technicians, or medical personnel. This study yielded some representative data for the device of this invention. Palpable myofascial trigger points are located by a medical professional or other diagnostic methods to design the treatment procedures. Some of these points, when manually stimulated, usually coexisted with that described by the patient as the location of their pain. In the FDA trials, a trained technician used the VAS, visual analog scale to assess pain levels of the patient, before and after treatment for each treatment and application of the device of this invention for approximately 45 minute treatment time every three days for the six week study period, for the FDA study.

For the visual analog scale, 0 to 1 is not pain or occasional slight discomfort. The range 2 to 3 is mild pain or annoying enough to sometimes be distracting and 4 to 6 is moderate pain which is distracting or cannot be ignored for more than 30 minutes but one can sometimes still work, in the 7 to 9 range, intense pain makes it difficult to concentrate, greatly limits activity and interferes with sleep, cannot work. For a VAS scale of 10 worst pain, or severe pain usually accompanied by difficulty walking or unable to walk which was noted in our clinical trials. A multiple baseline design was utilized where all subjects received the non active treatment mode for the FDA study. After stable base lines were achieved, for each patient then they were randomly chosen in sequence to receive active treatment.

For the patients in our formal FDA studies, we found ah extinction of pain responses less than the P≦0.01 level of significance per patient using the VAS scale. Some other clinical trials showed much greater significance in pain reduction. In another study which included twelve patients in a clinical trial, chronic pain, surgical recovery and post operative pain patients were included with a P value of P≦0.0007. The VAS value 0, no pain to 10, maximum pain was recorded before after each treatment session. Stable baseline conditions had been obtained up to a minimum of five days prior to the initiation of the active treatment condition for the noninvasive magnetic pain control device. No discernable side effects were noted at any time during and after treatment schedules. Presented are the pain reduction studies for the patients of the FDA trials. These give some typical results. In other clinical trials, treatment regimes were better designed for the particular patients and usually required less frequent treatment regimes than were designed for the FDA-IDE treatment protocol. This particular study was for the treatment primarily of lower back pain. The pain objectification system of this device and other means of pain assessment were also utilized.

The etiology of chronic back pain and lower back pain is pathophysiology is complex. Some of the most common lower back pain conditions are: (1) lumbosachral strains and sprains associated with fatigue, inadequate muscle tone, loss of ability to lift; (2) herniated discs and injuries of the bone, joint, ligament, or fractures; (3) myofascial syndrome (muscle spasm); (4) degenerative disease of the spine; and (5) surgical recovery from back injuries.

The criteria for patient selection were: (1) six months or more of continuous pain that had been largely unresponsive to other medical and/or surgical intervention; (2) all medical or surgical treatments within reason must have been tried and found to be clinically ineffective; (3) no use of any medications for at least two weeks prior to the study and during the study period; (4) no current use of a TENS device; (5) no evidence of any significant psychopathology (that is major affective disorder, schizophrenia, etc.); (6) palpable myofascial trigger points that reflected the patient's pain complaints when stimulated manually or by a small calibrated pressure instrument applied to trigger points; (7) pain limited primarily to the lower back, buttocks, and legs; (8) ability to sit for the duration of the treatments of about 40 minutes, and; (9) minors and women of childbearing age were excluded from this study. Four patients were selected and they agreed to participate in the experiment. All patients signed informed consent forms which indicated the nature of the experimental treatment. A procedure for randomization for the start of treatment was utilized. No other pain related or other treatments were used during this study period.

A multiple-baseline experimental design was used for this experiment these clinical trials. In this design, a subject's subjective semi-objective and objective pain assessment was first identified and measured over time in order to provide a stable baseline against which later changes could be evaluated. Baseline recordings of pain intensity were collected for the first five sessions. The device was attached but not activated. Following fifth sessions of stable baseline recordings, one patient was selected at random and the device was activated during their treatment. Baseline recordings were continued on the other three patients. Once a change in pain intensity ratings is observed for the first patient for at least four sessions, a second patient was chosen at random, and his/her device was activated while continuing baseline recordings on the third and fourth patient. If changes in pain intensity are observed for the second patient for at least four sessions, the device was activated for the remaining two patients within a four session interval. The patients were not told when their device has been activated. Experimental controls were demonstrated if changes in pain intensity occur only after the pain device is applied for any patient and untreated patients recordings remain relatively stable. The patients were treated identically in the both types of sessions. By this procedure, placebo effects were ruled out.

PATIENT A of the FDA trials was a 47-year-old, Caucasian male at 5′10″ and 162 lbs. Employed with an unremarkable medical history, he related the onset of his low back pain to a moving vehicle accident in which he was run over twelve years before treatment with the device of this invention. He had his first laminectomy of L4/L5 a year later. A repair was performed twice in the next year. Intermittent pain continued which was markedly accelerated when he injured his back during an exercise program four years later. After numerous non-surgical treatments, he had another L4/L5 laminectomy ten years after the first one and one year before treatment with the Rauscher device. The pain was described as throbbing, sharp, and continuous. It was located in the low back with left sciatica distribution. The pain required him to take numerous breaks every two hours while awake and would occasionally interrupt his sleep. Numbness of both feet had worsened during the past two years. See FIGS. 16a, 16b, and 16c. Vital signs were normal. Physical exam was unremarkable except for a number of trigger points in the low back and to the ankle joints and numbness to pin prick sensation in both feet.

PATIENT B of the FDA trials was a 59 year-old, female. She had a long history of chronic neck pain of 12 years and low back pain for 8 years which would interfere in her usual domestic activities. Pain was described as throbbing, aching, sharp, burning, and continuous. This patient had a long history of drug intake. Numerous therapies, such as physical therapy, massage, chiropractic manipulation, acupuncture, traction, biofeedback, epidural anesthesia, etc. had been administered to this patient without success. Examination revealed a Caucasian female, 5′8″ at 125 lbs. with normal vital signs. There were numerous tender trigger points at the base of the skull, at the right in scapular areas, and across the low back area. Neurological exam was unremarkable.

PATIENT C of the FDA trials was a 60-year-old, Caucasian male. Previous medical history included a splenectomy in 1943 and a broken leg in 1944 secondary to trauma. In 1970, he had a laminectomy but was pain free until he rolled a construction loader in 1981 which crushed four lumbar vertebrae in the accident. A Harrington rod was inserted in 1981 and surgery was redone in 1982. Severe throbbing, aching, tingling, and numbness in both lower legs and low back pain was noted since this time. Pain would increase with activity and be somewhat relieved with rest. Examination revealed a fit-looking 60 year-old male with a height of 5′11″ and a weight of 190 lbs. There was a general limitation of movement of his lower back with numerous trigger points identified. He walked slowly and with a limp and was unable to work. Gross neurological examination was within normal limits.

PATIENT D of the FDA trials was a 40 year-old, Caucasian male. He occasionally worked as a cook. He had a long-standing history of neurofibromatosis with no other significant medical history. Low back pain had been present for over two and a half years. Pain in the right leg was noticeable every day and described as aching and shooting with the feeling of cold or sharpness. Pain was reported to interrupt his sleep at night and to force him to rest approximately two hours, during the day. Ibuprofen, aspirin, and narcotics were used. A recent surgical attempt to remove a large neurofibroma of the right thigh was unsuccessful. Examination revealed a somewhat slow 5′9″, 175 lb male, having normal vital signs. Patient ambulated with a noticeable limp and appeared to be in pain. Numerous neurofibroma were present. A number of trigger points were found in the upper and lower back. A moderate-sized, right inguinal hernia was present. There was no gross neurological deficit.

From among the four patients, the longest period of chronic pain which they had experienced was twelve years and the shortest was two-and-a-half years. Three of the patients had had multiple back surgeries. Three patients were male, one female. The youngest was 40 years old, the oldest was 60. All were mentally stable and had sought and received medical and/or surgical treatments previously, without success.

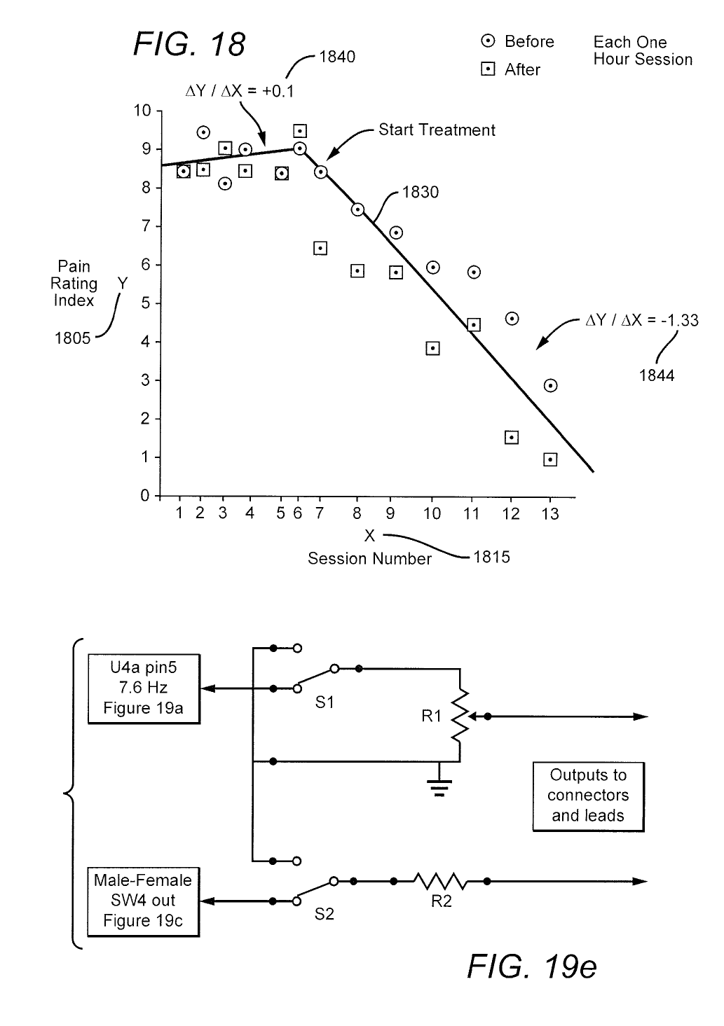

In FIG. 18 is displayed example data for the patient described in FIGS. 17a, 17b, and 17c. In the Figure is displayed the control and treatment data for the back and leg pain present from the time of the vehicle accident. A dot with a circle indicated the before treatment VAS values and the dot with a square around it indicated an after session treatment VAS value. Each session showed a decrease in pain from the previous session when treated with the device of this invention and during each session there was a decrease in pain from before to after that particular session. The subjective component consisted of patient reports in a structured medical interviews situation and their response a ten point pain assessment scale. The scale was graded from 0 (no pain) to 10 (the most acute pain they have ever experienced). The semi-objective component of the pain assessment consisted of trigger point determination. Over the first six sessions no treatment was applied and the patient remained unaware of this condition and his VAS scale remained between 8 and 9.5. In FIG. 17a is displayed the region of pain as assessed by the patient and is marked in black on the body form. The primary diagnosis was of sciatica. The pain originated in the L4/L5 region and progressed down the outside of the left leg and in both feet. Two treatment regimes were performed for this patient. The first, noted in FIG. 17b with the device of this invention utilized the positive polarity coil as C7 and three negative coils, one at L4/L5 and one each at the sciatic injection points. The first treatment was conducted for 40 minutes. The second phase treatment was conducted with the coil in FIG. 17b removed and with two minus coils on each side just below the left knee and at the inside and outside trigger points above the ankle and was conducted for 20 minutes. These treatments with the correct variations and placement of coils and levels of treatment, with the correct patient feedback were conducted over the next seven sessions, over a three week period, where for the active condition of the Rauscher pain reduction device. His VAS score went from an average of 9 to 1 and remained around 1 to 1.5 two weeks later for his pain from his accident. The lowest since his accident (follow up treatments were suggested and used).

This referred to those earlier treatment sessions, under the left-side arrow of the diagram in FIG. 18. The device of this invention's emitter coils were applied to the determined trigger points and to other appropriate locations. The device was turned on, and knobs were adjusted by the operator as if a real treatment was being given. However, during the non treatment the medical instrument was made inoperative so that no current was applied to the coils and no magnetic field was emitted. In the active treatment condition, the device of this invention was turned on and magnetic fields emitted from the coils. The coil or coils of the device of this invention was again applied to the painful areas of the patient; the device was turned on; and controls again adjusted. Only this time current was applied to the coils and a magnetic field was emitted. A least squares was fitted for both the before treatment session (circular) and the after treatment session (square) data points. We calculated the initial slope of the data points from the non treatment sessions VAS values reported by the patients in each session. A second independent slope was calculated for the active treatment sessions VAS values. This statistical analysis was performed in order to determine noticeable changes in the slope in order to illustrate the degree of clinical improvement, over time, during the active treatment session period of the experiment. A comparison was made of the non treatment sessions versus the active treatment sessions curves. Derivatives or slopes were calculated to derive a trend analysis for each patient. This induces the incremental change of ordinate, y vs. the abscissa, x. In the example given in FIG. 18, the non treatment sessions yielded relatively stable baselines with less than a 2% variation in slope of dy/dx for the control runs. FIG. 18 displays the before and after session treatment for Patient Se. Sessions one through six were conducted as a single blind real session to-achieve a base line. Then from session seven on, the active treatment modality was used for the treatment of his back problem as shown in FIG. 18. Normally follow up treatments are periodically used over the next few months to maintain and enhance the results, but this was not possible under the protocol of the FDA guidelines.

Four significant observations of these data can be noted. They are (1) That there are essentially no apparent differences between before treatment and after treatment VAS scores during the non treatment session portion of the experiment (2) That there are approximately zero slopes of the data points associated with the non treatment sessions, indicating that there is no effect of the non treatment with the device of this invention instrument in a non operational mode and little or no placebo effect (3) There are discernibly and progressively larger differences between the before treatment and after treatment VAS scores during the active treatment sessions periods of the experiment. and (4) That there is a progressive decline that is a negative slope in reported VAS scores during the active treatment sessions indicating a cumulative beneficial effect of repeated treatments with the device and procedures of this invention.

During the non treatment sessions, and even though the patients received attentive care, both subjects Sa and Se showed a slight increase in reported pain level. This was quantified by the very slightly positive slopes during this portion of the experiment. Some patients stated that travel to the clinic, even for the three who received a ride increased their pain level. The before treatment and after treatment VAS numbers did not deviate significantly from each other, during the non treatment sessions portion of the experiment. However, for the active treatment sessions, each of the after treatment VAS values were always less than each of the corresponding before treatment scores for each of the four patients. No patient appeared to recall what was they reported as a previous VAS score.

The averages for the FDA clinical trials reflect a continuing reduction in pain each time an active treatment session was administered. Another statistic utilized for comparison purposes, was the standard deviation of the scatter of the VAS values during the non treatment condition. The standard deviations for both non treatment sessions and active treatment sessions were calculated separately for each patient.

Comparison was made statically between the slope of the curve between the before or non-treatment condition to that of the start and completion of treatment. These slopes were derived from least square fits. We also plotted a line through the non treatment condition points on the VAS scale and the line plotted through the treatment points match the last non treatment data points. Before session and after session points were plotted and a line drawn through them. Slopes of curves were derived from least squares fits to before and after treatment, in which session beginning and session ending points were analyzed together. The standard deviations for the active treatment sessions were substantially greater. We interpreted this as reflecting the beneficial, pain-relieving effects of the device of this invention treatment during most of the active treatment session.

A two-tailed t-test comparing non-treatment sessions versus active treatment session VAS values was made for each subject individually. The p values for all four subjects ranged from p<0.03 to P<0.01. The small p values were obtained for the patients with the most treatment sessions and the higher p values from patients with the least treatment session. Combining the observed p values for all four patients and making the assumption that they represented four completely independent observations, the combined probability of observing these four low values is about 1×10⁻⁷. This data is given in Table 3.

We continue the least squares line from the non treatment to treatment condition in order to analyze the proper relationship between the two conditions to demonstrate the relative difference. A six month clinical follow up showed that three of the patients, Se, Sa, and Sb remained relatively free of pain and that patient Sm remained in pain at about a VAS level of 6 due to the continued presence of his hydrocele which he had surgically repaired. Further treatments were not made because the FDA study was completed. Proper use of the device of this invention was however indicated.

The sample size for the FDA study was limited but demonstrated positive effects in pain reduction and no side effects. Other clinical trials have also demonstrated significant positive effects and no side effects sometimes with greater levels of pain reduction due to a personally designed better treatment requirement. Presented here are a few more of many example cases of recent clinical trials.

A female patient, Us, 71 years old, 5′3″ and 123 lbs. exhibited severe osteoarthritis of both hands. A treatment regime of four treatments over two weeks reduced her pain level before treatment of a VAS value 5 to 6 to after treatment schedule of 1 to 0 and with a very significant increase in flexibility and ability to grasp objects with her hands.

Treatment of the vagus and phrenic nerve of a 22 year old male, Jm, 5′11″ and 158 lbs. with an unremarkable medical history. He complained of pain in the vagus and phrenic nerve region as well as the fossa concavities of the iliac region and of the pelvis for five years and rated his VAS pain level at 7. After 3 treatments of these areas of his body, he reported a VAS value of 1 to zero.

A male patient, Mr, 53 years of age, 6′0″ and 175 lbs. broke rib #3 and sustained a sprain, also with muscle tenderness in #4 and 5 on his right side. He was properly treated for these injuries sustained in a fall from a ladder and remained in bed for three days and experienced poor sleep. He received five treatments over a week and a half and was able to sleep through the night and experienced less pain with each breath. He rated is before treatment VAS rating as 7 to 8 and after treatment as 2 to 1.

Another male patient Pm, 5′7″ and 136 lbs. with an unremarkable medical history, presented with tennis elbow or posterior cutaneous pain over the lateral epicondyle of the humerus. He rated his pain as VAS 5 to 6 and sometimes up to 10, and after two treatments over 5 days, reported greater movement and an after VAS value of 2 to 0.

A female patient, Rf 54 years of age 5′6″ and 175 pounds has suffered sciatic pain on and off for 23 years and was particularly bothersome over the last three years. Treatment consisted of four treatments of the L4-L5 region. She reported a before-VAS rating which was between 7 and 6 and after treatment she rated her VAS value between 1.5 to 0.5.

A female patient, Mm, 5′6″ and 118 lbs., reported pain in her sacroiliac and the anterior surface of the iliopsoas muscle for over five years attributed by her to by stress and injury. She rated her before VAS scale as 8 and after three treatments she reported very good results with a VAS of less than 1 to zero.

Patient Pj, an 86 year old male, 5′10″ and 152 lbs. experienced an ischemic event thrombosis of the Broca's area and remained in intensive care for 12 days. The prognosis for his right side paralysis was poor. A series of treatments over one and a half years lead to no apparent paralysis and he was able to walk up to eight miles a day. No physical therapy was used except walking. However, although his speech improved, it did not return to normal. He rated his mobility from ability to walk less than 50 feet to normal walking ability and no associated pain.

A male, Ad, 6′4″ and 168 lbs. had suffered pain in his right heel from a bone spur on the calcaneus and rated his pain on the VAS scale as 5 for over eight months. After one treatment, he rated his VAS value of zero

A male patient, Es, 49 years of age, 5′11″, and 168 lbs. presented with a left sprained ankle from falling down his back stairs. Present was significant edema and black and blue discoloration. Proper medical diagnostics showed no broken bones and an ACE bandage was applied and use of a cane. Three treatments were conducted over eight days. Swelling was reduced and the before VAS score of 6.5 was reduced with the treatment of the device of this invention to from 1 to 0.

A female patient, Ew, 37 years old, 5′2″ and 138 lbs. had suffered a significant lower back injury at 8 years of age. She performed heavy lifting in a move and suffered a skiing accident that hospitalized her for 10 days in traction and she remained bed ridden and house bound, unable to drive for five months suffering from significant sciatica. She showed a compressed disk between L4 and L5 and a L7 rotated disk. After a series of treatments over two and a half years she was able to return to work full time. Before treatments she did not sleep well and rated her pain of a VAS value of 9 to 10 and after the series of treatments she rated her pain from 0 to 1. She returned to a full exercise regime including weight training. She expressed her pleasure of being pain free after so many years. Follow up showed low or no pain in most patients.

A female, Ee 53 year old 5′1″ tall 158 lbs. woman tore her right knee anterior cruciate ligament during a running accident. Before she had the required surgery, she could not move her lower leg to the left of vertical and was on crutches and unable to work. She received treatments over six weeks. Before treatments she rater her VAS value at 9 to 10 and was faint after the accident. After the accident and treatment with the device of this invention she was able to return to work and prepare for surgery and rated her pain between 0 and 1, when she walked without a cane.

Subject, Wb, a 53 year old male, 5′10″ and 152 lbs., presented with past operative surgical intervention from a pararectal abscess having a temperature of 104° before surgery and 101.5° at treatment. This subject was in general good health. Surgery had left an open wound approximately 65 mm wide and 70 mm deep at its center in the left gluteal region near the anus. The subject rated his pain level VAS at 9 and was restless and was unable to sleep without waking every 20 to 40 minutes even though he was given pain medication. Upon a series of treatment over the next five days with negative polarity coils directed to the site of surgery, he was able to resume a relatively normal daily regiment and after the first treatment rated his pain level at 2 and after five days reported a VAS of zero. Recovery was rapid and the wound was packed and kept sterilized during the recovery period when needed

Subject Vr is a 46 year old male, 5′11″, 163 lbs. in excellent health. He presented with a deep cut approximately 24 mm in length with significant blood loss was evident on his third digit of his left hand of the proximal phalanx. Upon cleaning, soft tissue damage was observed to occur to the bone. The cut was cleaned and the edges were made contiguous and loosely wrapped in an anesthetic dressing. Two negative electrodes were placed dorsal and palmar over the site of injury. Treatment lasted four hours. The next day upon removal of bandages, a thin slightly red line was evident, no bleeding was evident and upon stretching the wound, the skin remained intact and no pain was present. Many other clinical trials at four major clinics are documented with similar significant results.

### VI. Circuit Diagram and Description

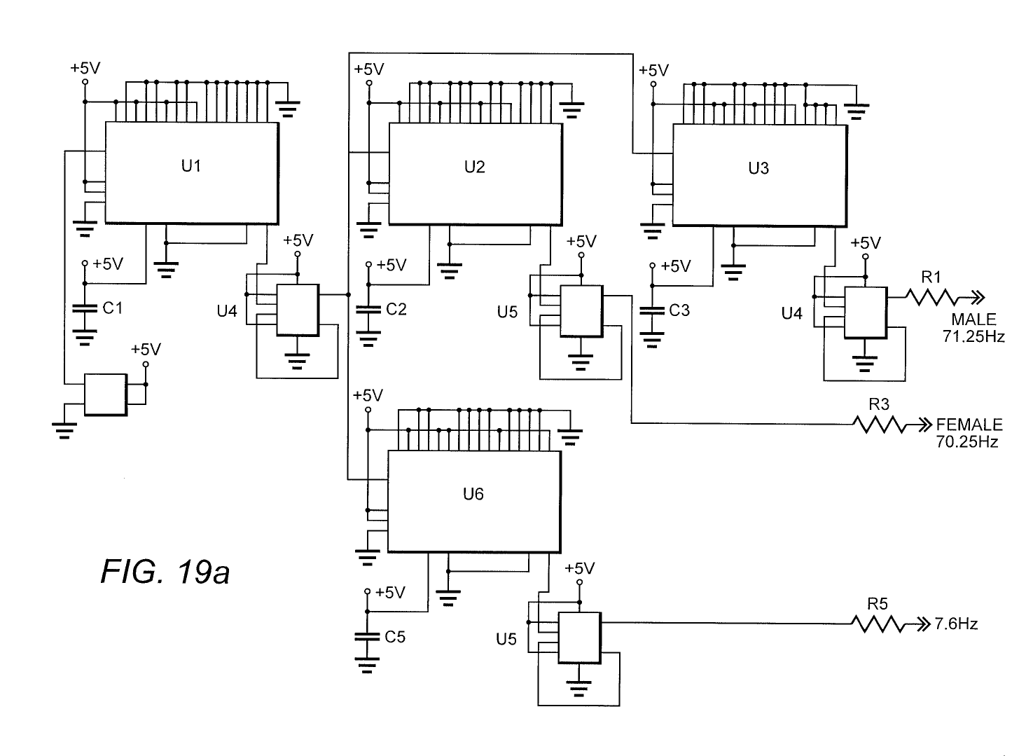

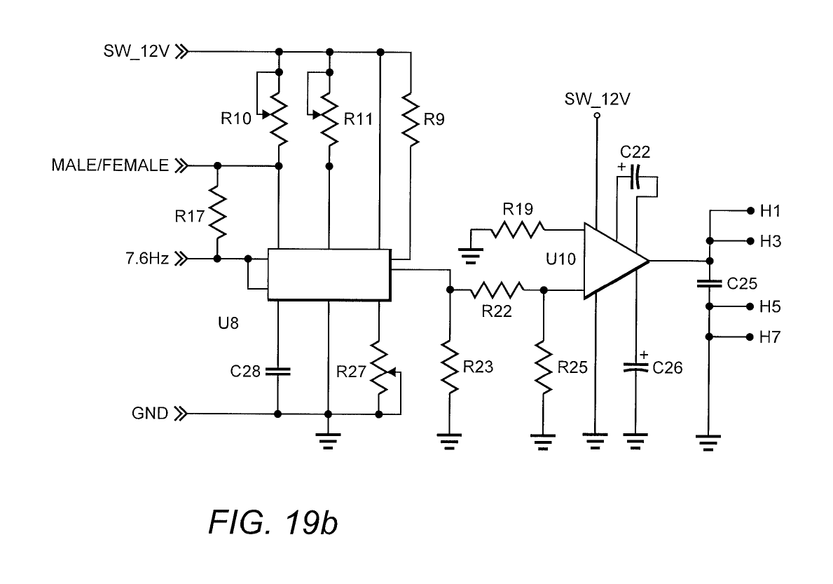

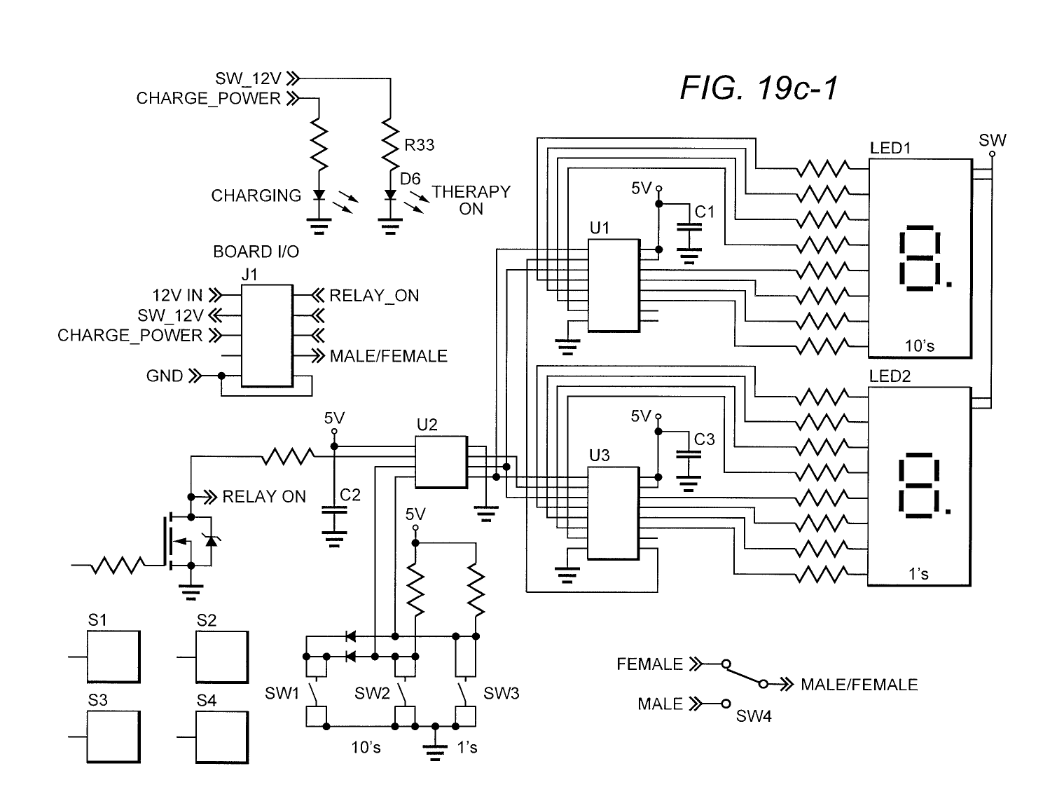

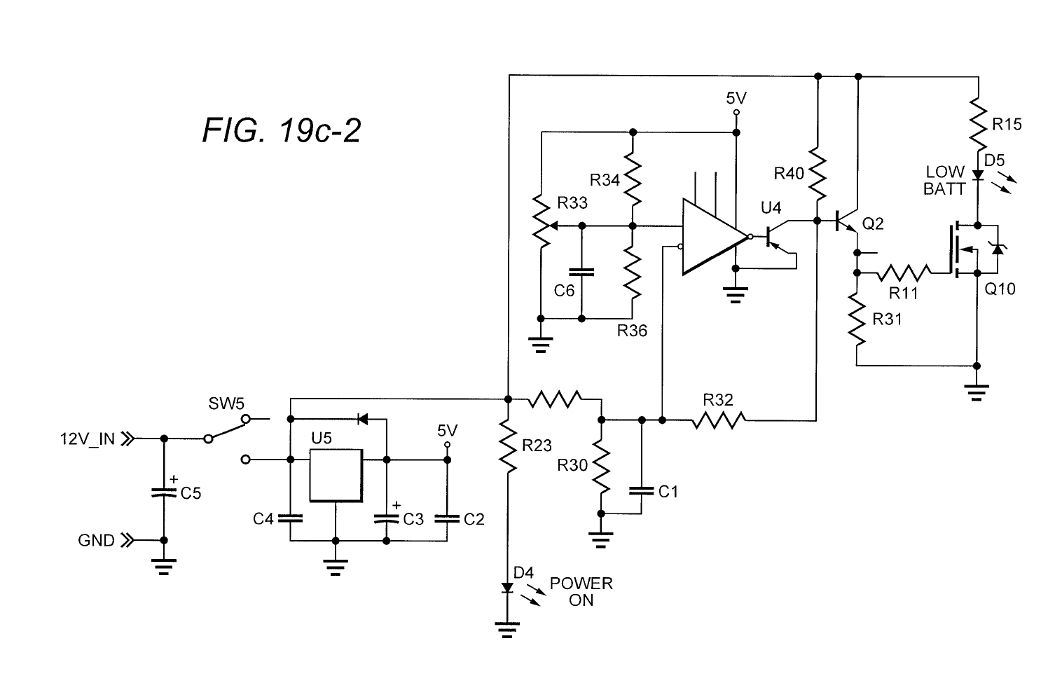

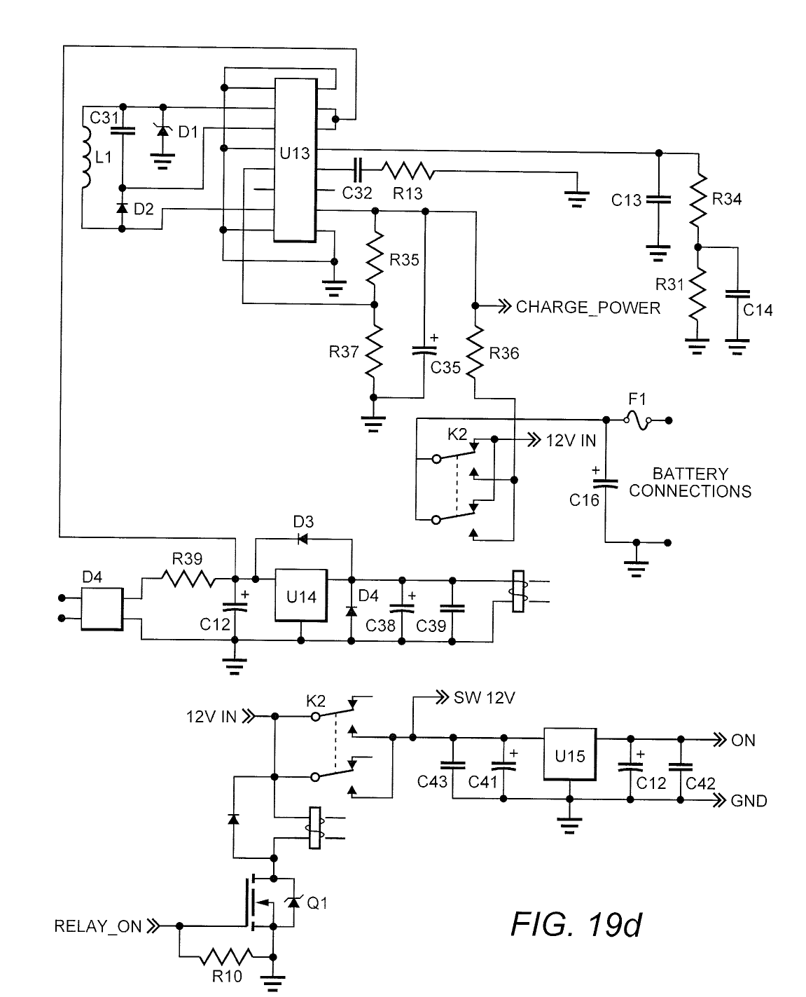

The nonevasive pain reduction device can a battery powered, FIG. 19d. The battery is charged from a remote step down converter (18 volts DC, 750 mA), powered from the line source. The device does not allow for simultaneous operating and charging. Charging the battery is indicated by the charging LED (D1) and current limit resistor (R2). The charging circuit consists of an input rectifier bridge (D4) and current limit resistor (R39), these components allow for correct input polarity and current limiting. Charger input voltage is detected by relay (K1) and when energized begins the charging process. The charge controller (U13) and associated components, (L1), (D1), (D2) and (C31) form the charge switcher and the regulation components, (R35), (R37), and (C35) control voltage. Timer components, (C32), (R33), (C33), (R34), (R36), and (C34) control the current charge rate. Rate of charge is set to 50 mA and recommended highest charger voltage not to exceed 20 volts.

The battery is protected by fuse (F1) and resistor (R36) the capacitor (C36) is for and noise suppression, see FIG. 19d. Also for noise suppression are the capacitors (C37), (C38), (C38). Minimum charge time is 2 hours and cannot overcharge. Upon removal of the charger, unplug it, relay (K1) returns to normal operation and the device may be operated.

A. Therapy Timer and Power Switching Nonevasive Pain Device.

Power switch (SW5) turns on the nonevasive pain device, see FIG. 19c, as indicated by the power LED (D4), and current limit resistor (R23). Battery condition is determined by comparator (U4) and associated components as a precision threshold latch and performs the low battery lockout function and is indicated by low battery LED (D5), also disables control line output with a clamp transistor (Q10, and associated components, (R15), (R11). Power regulator (U5) providing 5 volts to microprocessor (U2), the timer and associated components, displays (LED1), (LED2) and display drivers (U1), (U3), along with programming switches, (SW2), (SW3) set the duration of treatment. Start switch, (SW1) begins the countdown timer and also supplies a control on voltage, 5 volts to the (relay on) control line. This control line switches on relay (K2), with (Q10 and (R30) providing 5 volt power (U15) for frequency divider, and 12 volts for the modulator and output driver. Therapy on indicator LED (D6) and current limit resistor (R33) signal the device is active and will remain illuminated until timer has reached 00 time.

The Rauscher device incorporates two precision timing oscillators, see FIG. 19a, that when combined form the timing sequence. Frequency stability is achieved as shown in FIG. 19a. The clock timing circuit is power by a regulated 5 volt source. This supply voltage powers the master clock and a series of high speed frequency dividers. A master clock set to 32 MHz derives the common core frequency. This frequency driving the dividers then divides the clock into three timing frequencies. The frequencies are 7.6 Hz and selective and 70.25 (female) and 71.25 Hz (male). These frequencies are gender specific and must be mixed correctly.

The frequencies are buffered and coupled to the Modulator Drive Control through the low impedance of 100 ohms. This achieves a square wave drive voltage (4.8 volts) from all output signals. See FIG. 19a.

The frequencies utilized in the device in this invention are those that are characteristic of a normal healthy body. This sequence is used to time and modulate a gated variable frequency triangle waveform generator, and a variable gain amplifier. The basic design is shown in FIG. 19b. The circuit is divided in two sections, the waveform generator and the gain stage.

The initial oscillator frequency, see FIG. 19b, is set to 3040 Hz. The frequency is set by R27 and timing capacitor C28. The triangle waveform is nonsymmetrical, the leading ramp is 33% of the duty cycle and the trailing ramp is 66%. Symmetry is set by R11 and the inversion is set by R10. The duty cycle is then modulated selectively, by the timing sequence. Both high and low timing frequencies are intermixed by R17 and drive the frequency modulator. The oscillator frequency is modulated 10% by the timing sequence centered on the initial 3040 setting. The variable gain amplifier is gain dependent on signal density. This produces a cascade ringing of the signal. This signal is delivered to the therapy area by a number of coils (600 Ohms) to provide direct magnetic stimulation to nerves and soft tissues.

### VII. Pain Objectification and Analysis

Objective pain determination was made by placement of cutaneous electrodes placed near trigger points and a small current below sensorial threshold was applied. The current level was increased until the patent exhibited a startle reaction and reported a sensation of “tingling”. The level of current flow at detection threshold greatly increased as pain was decreased by treatments with the device of this invention. Blood flow monitoring demonstrates that there is increased flow in lower limbs after treatment with the device of this invention in cases in which the patients had restricted blood flow.

The pain objectification output signal can be achieved in one embodiment of the device of this invention by the use of two output frequencies from the divider schematic, see FIG. 19a. The first of the two frequencies is derived from pin 5 (U4A) output from timer or equivalent to the cutaneous electrodes goes through 100Ω fixed resister and then to the connector jack to the cutaneous electrodes. The second of which goes to a level control device such as through the 50 kΩ, ten turn potentiometer having a digital readout (1-1000 scale) which then goes to an appropriate connector such as a phono jack, BNC or other connector into the cutaneous electrodes. Other scale reading systems can be utilized to analyze current and voltage input to the cutaneous electrodes and give an output value to be recorded such as on a computer, etc. Pain objectification can be accomplished by the use of this part of the device of this invention. The dividers produces the 7.6 Hz signal and the 70.25 Hz or 71.25 Hz signal detailed earlier here including the 5 volt power supply specifications. The output signal from the upper frequency is fed into a 50 kΩ ten turn potentiometer. Other electronic potentiometers can be utilized. The read out scale of the detent position of the dial corresponds a 1000 digital scale selling of this device which is impedance matched to approximately 1.4 MΩ resistive load of human dermal readings by use of cutaneous electrodes. The stimulus for the pain objectification device of this invention is not felt are barely noticeable by a person who is not in pain. For a person not in pain, full voltage and current at 1000 scale reading is usually not noticed at all by a person free of pain. If a person is in significant pain or in moderate pain, they report the perception of the tingling sensation at a detent reading of 200 to 300 for example for a VAS report of 7 to 8. They may report the tingling sensation at 500 for a VAS of 5 to 6. This is fairly uniform across patients. The pain objectification part of this invention can be utilized with the device of this invention, or to assess the level of pain independently to assess the degree of pain being experienced under any condition such as in the claims of injury. Such a configuration of the output is about 1 to 3 mA and can be used for this unique and novel application in pain objectification.

Two or more cutaneous electrical signal pick up electrodes are placed on either side of the region in which one desire to access information on the stimulus tolerance level. A small AC signal of a frequency intermix is generated between the electrodes. An AC signal preferably in the range of say 3 Hz to 80 Hz is preferable but in some cases a DC signal which can be utilized. Current flow is not perceptible to a person free of pain. Increased sensitivity occurs when a person is in pain. Electrode placement can be made near the region of person's report of pain or near but not at the locus of pain. The cutaneous electrodes can be placed on any part of the body preferably not the head for pain objectification. To objectify pain the current of approximately 1.2 to 1.5 mA is turned on at the skin surface. The maximum voltage is 5V and is reduced to 1 Volt or less. The level of stimulus perception is then recorded on a digital scale of a multi-turn potentiometer dial which is marked with a scale of every digit, or other useful scale the lowest stimulus is at zero and the upper level of stimulus such as at 1000 on the dial detent position. The level of stimulus perception is recorded at which the person perceives the stimulus, which is often accompanied by a startle reaction and their report of sensation. This stimulus is not found to be unduly objectionable by patients. This level can be noted before and after treatment. It is noted that there is very good correlation between the level of external electrical stimulus response and the level of pain reported on the VAS scale. Also the level of tolerance of the external response and the person's report of that response is strongly correlated and reduced so that a person who is pain free from the use of the device of this invention will not perceive the maximum level of stimulus or 1000 or equivalent on the dial detent position. The signal is a two or three frequency signal having beat frequencies of 23 Hz and 830 Hz. The diagnostic procedure of the CNS and PNS can be made by a DC induced current flow and coil pick up placed between the two or more cutaneous leads. In the case of a DC signal across cutaneous electrodes, the current is about 25 μA but this method is less effective.

Diagnosis of the condition of the functioning of the biologic system can be made in terms of the number of Fourier components observed. These are affected by the effective duty cycle of the correcting emitted field. Measurement of nerve impulses in vivo, muscle contraction and bone and other piezoelectric response can be easily distinguished in the frequency domain. Nerve cells give off a short duration discrete digital like burst where as muscle contraction and bone flexation can be readily identified by the longer duration impulses. Muscle burst impulses lie between neuronal activity and muscle and bone flexation and the piezoelectric response of collagenous material. Bone and muscle potentials have a much slower rise and decay time than neurons. We can shape our signal through the proper electronics of this device and the proper coil configurations to effect the proper signaling to affect the desired tissues.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a living being, comprising:
producing a first signal having a first frequency and a second signal having a second signal that is different from the first signal; and
superimposing the signals upon a tissue of the living being in a manner that is not phase locked, and at physiologically acceptable intensities and duty cycles such that the signals entrain the tissue.

2. The method of claim 1 wherein the living being is a human.

3. The method of claim 1 wherein the first and second signals are selected to produce a plurality of intermodulated additional signals,

4. The method of claim 3 wherein one of the frequencies is selected from the list consisting of 7.6 Hz+/−2 Hz., 70.25 Hz+/−0.25 Hz, 71.25 Hz+/−0.25 Hz, and 3040 Hz+/−10 Hz.

5. The method of claim 3 wherein both of the frequencies is selected from the list consisting of 7.6 Hz+/−2 Hz., 70.25 Hz+/−0.25 Hz, 71.25 Hz+/−0.25 Hz, and 3040 Hz+/−10 Hz.

6. The method of claim 1 wherein each of the first and second signals are electronmagnetic.

7. The method of claim 1 comprising using a multi-coil array to produce the signals.

8. The method of claim 1 further comprising applying the signals for a duration of time sufficient to trigger a subjective reduction in pain in the living being.

9. The method of claim 1 further comprising applying the signals for a duration of time sufficient to trigger a subjective mood improvement in the living being.

10. The method of claim 1 further comprising applying the signals for a duration of time sufficient to treat osteoporosis in the living being.

11. The method of claim 1 further comprising applying the signals for a duration of time sufficient to enhance cardiac functioning in the living being.

12. The method of claim 1 wherein the tissue includes Purkinje cells of the living being.

13. The method of claim 1 wherein the tissue includes cells of the hypothalamic pituitary (H-P) axis of the living being.

14. A method of treating a living being, comprising:
producing an intermodulated signal comprising at least two electromagnetic signals selected from the list consisting of 7.6 Hz+/−2 Hz., 70.25 Hz+/−0.25 Hz, 71.25 Hz+/−0.25 Hz, and 3040 Hz+/−10 Hz; and
applying the intermodulated signal to a tissue of the living being at physiologically acceptable intensities and duty cycles such that the signals produce a resonance lock with the tissue.
